SEQUENCE LISTING (17 sequences)

SEQ ID NO: 1 - DNA, 4186 bp, Homo sapiens
cgacggcccgggctggtaaaaaatatgtgtgtgtgtatacttaaataaatatatatatataaagtcttggacttattttcacctagcctggggcatatcgtggatagtttcaatcgacaaaacaggcactctctggcctaggagtggaatccatcctcaattttcgcaggctcctcacaattctctatttggaagaagtgtccctctcctccccttttcttttcctcctttactcagcgtcagtccccgcagccatctcctccgaccctttttgtctacgtcccagcgtcgcgaaccacagcggcggaggtggagcggggagaggcgttaggccgggcggctaaaacgcgccgttaaagtgggggagagattgcgcggagcccacgcgatccctgggacgccggagacaacggggctcttgggaaggcgcggagcccggggaagccggggatgtgcgcgtgagccgtgcccgcagggtctccccgcctccgccacctttcttgggtggctctccgcctcgtcccctctccgagggccgttggtacattcctagtgactccaagcgcttaaaaggggcccgggagggtgaaccccacagatctgaacctgatttgtgtgtgcaccgcgtctccagcgatcccggatccactgcgctgccaggggcctgggggtgggtctcttgctgtctctgcgacgacatccttacgtttcggcactctaatgctgggtttgtgcgtgtgtgtctgcttagcggtctagcgggctgttaggcctccctcgcccccagctccttggtcgctcagctcctccaccgcagcccagcagtgagacgcgcgcgcagccagctccccacgagatggaacagaccgaagtgctgaagccacggaccctggctgatctgatccgcatcctgcaccagctctttgccggcgatgaggtcaatgtagaggaggtgcaggccatcatggaagcctacgagagcgaccccatcgagtgggcaatgtacgccaagttcgaccagtacaggtgagcgcgctgcaacttcaatgccaagcggcgcccactcgccccgtctgawggaggaatggacgtgggggacgcaccccaaagtgactggctcgggaagctcgggccgcgctgcgtccgccgctcgccccctcgctggccagccgcggggctggcacgactcgcttggggctccgcggccacagcgcaggcgggaaggtggatcctactggccttgctgacctccggccgggaacggcggacaggcgccgggaagggaggccaccgcgcgtctgcgagattttctgttctcttctcctccgcaaacccctccgcttctaaaccaccagtcttcctgcccctggcctcgggagcaatgcccacactcccaagccatcgttaaccccgggcgcggagattcggagagaccgcactcaggttcgggccctcctgacaccttaaggaaccgtctggccggactagcagagggtgctcccaggggagcactacatcagccatcgggagaagtgtgtgatgttgaagaagacaagtcacccgcgtcagttctccatcactgcgccttcggtcgcctgcaaccgggcaccttatagcaagcatttttactcatgttaaatacgtcttgtggccgtcagctgccactacggaatttctgctgttccctttcccaaaccaatttaactaacttccccgactgtgggtttattcaaatccactgccaagtttcgcagattaaatattcgaccgtcgtttttttcagcccccaaatgatctgatttatcttatccttgcttctgacgatctgtaatgtaaattaaagacccctacttcccagatgtacttgagtgaggtgccctggtaagctggcttttttgcagccccggcttggtgattaagtcgaatctctctaccaatcctccggggccagaggtaaaatttaaccaccccgaagtgtgagaggttctactttttctgagcagttagtgcgtcagtctttgtggttttttgggtttagggttaggagaaaggggtaagtgaaagaaaagaataaggaaagaagtaagtttgtttttcttgggagcaggttcttttagtcactctttcaacatgtgggatgtttcgtaaagttttgttggcatgggcggtttcaactgtattttaggatttgaaatagaaaaagtgaagaccactaaggatcccatttttcatatacaagtgacttgaggaaattcctttggctcctatcagcttagttcatggtagagcccttctgtagccgtgaaagctagcagagagtctagcttttaaattttcttgcatttgctccaggtaattgaagacttgttatgggacaggtactcttccagacacttgacacatatgaggaatggcaggcaatgaagaagttgaatgacttgcactaggtcttctatcgatagcggctccaggctcccggggttagcattgttgttccagagcctacactctaaaccactgttaaaccaggacaggttgtaacagtttttaatctttaatctttagaacaagtgtatcttcaaggaccttgaactagtctcgattgacaaactctaaagcttttgaagtgcaaccagctgggttgatcggtcttttcacagatatctgaggtacagctcattaacataatggatgtattcccgagggggcagttgggggaagacaggatatgggagagaagtcaattctacagtattttcttgcattaaataggtgcctaacgctgtgaaggattctgttctctgactttgggagggccagtgcagaagaatcaagacagaatatgctcccgtgtgggccaggctgtgaattctttgaaagaagctgcagagaagccaacaaagattatcctaagacttctggaggaagaattaggatccttctgcaggaactcagtttactgtgaattgaaggttgtgtacacagcttcttgttagttcataaaggctacaaagtagggaaaactgaggctaaaaagcacttactaagtaactccttctaggatcatgctcttcggacacctcggggtggggaggaatgggacaggatggaaggggcatgctggacaagagttaaccgatgacgaagggaaaaaaaactcccaaaaaatggagtaaagaccccttttctttaacatgacagtctaaatgaatgtcataattatgaaactttgctaactagatgctgtcaacagtacaccttggaccaatagaaactttgacgttgattttgcttttttcacagtcgtggtcgtggtttgcagtttgttgttggtagtggcagtggtggtggttggttgtggtgagtgtgtctgtgtgtatgtgtttgccagttactggtttctcctaattgtaagaggtttagagggttctttacttcactaatgtagtcttctttgttctggtagttagatcagcccccacggtatttgtggaagacttttcattataagggcttttgaatgtggtttgtgtctaggctttagtaaaactaagaattctagaacagctatttaatgaaaccttgtttctcaagtcttgatttagctacaggcagaaaaaagaaaaaaaaaaaaacaatagcaacattcttgcctcaaccagtaatgcactttggaaatactttctctgagaaacttactccatcagtttgaggctgtcacattgagaaacatgcaatttcggtttttttcctgttatttttcccgtgccattaattaaaactgagcagctacctctgtaaatgcagcggttcaaaatgaactgtgaaaaaggatttttcatttgtgttggatttctaattagaaaactgcattcaaagttgcttagaggtggaaatgttaacatttgtgaataaatggtacttggtgacacacttattttttccagtgcttgtttcagcaacgaacttaaaacttctgaatatattgtttttttttaggtatacccgaaatcttgtggatcaaggaaatggaaaatttaatctgatgattctctgttggggtgaaggacatggcaggttaaaataaaytattcattcacagatatttattagagcatgtttttctagcatatgaaaaatatagtaccaggctagccacaggtcttacaagtagtaacaaatctca

SEQ ID NO: 2 - DNA, 2739 bp, Homo sapiens
gagacagtggttcactcttgttgcccaggctggagtgaaatggcatgatctcagctcactgcaacttctgcctcctgggttcaagcaattctcctgccttagcctcctgagtagctgggattacaggtatgcaccaccatgcctggctaattttgtatttttagtagagatggggtttcaccatgttggttaggctggtctcgaactcctgacctcaggtgatccactcacctcggcatcccaaagtgctgggattacaggtatgagccaccacacccagccctcattttctacttacttgttcacaatgttccctaatcccaacaaaaattatcaatggaattagcacagggcattcaggcatacactacacatattaaaaccagcccccaaaatgaatagtaaatcaaatgaggtaagtaacccccttaagttaaccatactctgttttctgtgcagtaaaactttatgtgaaattgtgtttacacttgacatttgtggtgtttggggtgtgtaaacaatcgtgggaccaaagaccaaaggatggagattggaggctagaaaagcattgcaaattcagaaaatttcttctctacytcaagttaattctttagtttctctactgaaaaggcaccttattctgatcctgggaaacttagctccttaatttatctttaggtttcacgttgggcaggcttattatcaactaagtattactctcttttgaaacatacctttctcagccttgagaatggtaaaagcaataatttagatccatttttwtgaaagaaaacatgaatttataagtgtttggaaagttaggtctgtcgagtgcatttatttactcatatggaatgaagtgtttccattcattctctcaatatttttgaaagcacattatgtccagagtgggactagactctgtggatttaggagacaaattctttttatggtacatatataaattaataaaattcaatagatgaaatgaatacccaaaatgtatatagatacatatattttaattaaccctgtaaccacgactgcttcatatacttaaatactattcactctgagattgcataaataaatcctttgtttcctcaaatccaattaactttttcccaccaatgttggaaggtaatcacaggtagagcactgtaaattgacacaatgttaacattttatatacagtgtaactttaggatggtaatacacatatagataccttctctcccacagctccccaaaggaaggagaagagacaaaagccttggggaaaaaacattaaatgttaagtggatcttctcagtgtggtacaattactatggatgattgtcttaatcctttctaaaattctctaatgtactttcataatcaaattctagcccagtttaagtgagttatatgtatttgggggaagataaagtgttgctggttttgtgtttctgttatcaaagtagtataaccaaagttaaaaaaactgtccattagtaggggtatgcatcaaaattgtaccttttatatgaattgatatagatgatatattattactttaccctgaacattgttgaactgtaagttgataacattttaaaaattcagctgaaatggattttggctggggagatagcttattgacagtatgcaggtgtcagttctgggaagactgttactgcaggtctggatactagaccatctactgagttcagttgtcattttgtgtacatatagcagtattcatgatcataccaactcccactgctttctgaagatgctacagggaaatctaaaggagacattatttgcctggcctgacaaaaaatccaatgagatggtcaagaagtctgaaagagtcttgagggaaaaccagtgtgcctacatcaatggtaacatcttattctttttctaagggcaagtgggaagaaccatgacctttggtctgaaatattggttacttacactttttatgcagtagaacctggatcatgagtcatgattcaaaatacttgggaaatgacttaaacatagcaggtctggttttttaagcctttgtatcttggttggaatcacttattgaaaacatatgcatgtagttcagtatccactgaaaagtcatttacttgccaagacttaagacagttaaaataaaatatgaatttacttcagtggagtctttcaacaaaagagatcaagttggaaacattagccttgtgttagaaaatagaacagaatgttctgtaactaattaagaatgctagctagtctctgtttttctaaaatacatatagcatacctaaaaagtatcctcgagggaaattttacaaagcacgttattggccagtgatttatacatatcttaccaagaattccttatcttttctaaatgggatatgggatagccaaatacttttcaagagagcagtgattaatttccatattcctaaaaggaacagtaacttccaaaaagctcattaaaatgtatatgagacagatagatagttttttattcccttttacgtaggaggttgccaacacataggaagattaaacaatttgtccaaggttactcagcttttaagtggcaaccagaatttgaacctaagcaatttgattgaaaaacctatgttcttcatcagcatgtaccccacctatataaagtattatagagctttgggggatttttttttggcaaagcaagtttttttctccataaatcttactggcctcgatatttgg

SEQ ID NO: 3 - DNA, 2995 bp, Homo sapiens
tcttcctcagcatttcctgccaacccttttctggtttctgtttcttcaacctctttttgttttatggtcccttttatggtattttctcttatttctaggaaaacattaatgataggtttttgaacacttcccctgagtagtctctgttgcaggttgcttttttgtttgctctgccctctgtctttcatactagaggccttgctcaaatgtctggtgaccttggcaatgtgctaatatttaaaaaccaagaacaaaaaaattaattggtaaccctgaacatgagtggagcttgtcaactggatttcactttttgatagtctggcagagactttttcttcaggaacttccagcgacaatatctttaaagcttttttcttgagccaatcagattctctgagaagttgcttcctatcttctgcccggaaagaaaaatcttgtctggcagcattctcgcagctaggtgatgactaaagcagggatttttaccattcagtatggaagcttttgtttaattcacctgtttttcacctgatttccatcagtcccaagttgatctcttacactctttctagagactagaccttactctgctgctgaaaagaaagaagagagagaattggtaaaagggttttgagactctacttcataaacaagctttaaaccagttcttatatctacagcctatctttaccctcatttccagaagtacctggtgctgtcaattcctgtgctatttggagtttcaagatataaatcaggttatttcttggttttctccatggatagcttaggatttagctttctctgctctgtaaagctatccatcttactttctaagctttcaaatttttggtagggttttctctttccctatttggctttgtatattgatgtcttttttatttttttactgccattggagagggttttcaggaaggagcagaggctaatgcatgcgtgtcacttactatctttaactgcaagttcaattctaattaatatctattccatttgcatcacaattgattcatgatctcagatgactcacagagattttacctacagctgttatctaggctcctttatctatttttccttcccggatattgtttagattccattggcttacatcgagtagagaacatcagccatacggaacctgctgtgagccttcacttgtacagtccaccttttgatacatgccatgcctttgatcaaagaacaggacataaaaacaaagtcacaatgacattccatagtaaatttggaatcagaactccaaatgtgagtataatttcttctaggtttaaatgctctgtaggttggcactgttacactgcatgtatgcaaagaatgagatgagtcttaattcatggataaggcccaatatatcttgattgttttatttaactttagcgactatcttttcaatttatttttaaaaacttattatgttagatccaaacattagttgagaaatttcaaaatgtatgcttgtttttaaagaaaatgttttcatttacttgggaaatgagagtgaaaactattatttgatataactgttactgccatggcttactatgcataggtctaatttgctgggccaacagggataagttgcataattaacctttgtaaaggttattggctgatgatataactaaagcaaattgtctgttttgtttgccaatgttgcttattcttagagttgttatgccatttataccacaaataagaatagactatactaacatgttcctccttgagaaacaagcatttcctaccaaaaataaaaaggcaaccaacactaatatagcagggcagtcttctacctctgaaataagtttgtcatttagaggaatagtctttaattatcagaatatccagaaatagacaatgtgtgaaaatggagacccaagcaatccttgagtcttaatgaaagcgcagcctctgagggaagccagggcagaattttctcattatcgctccgcttcatctttatcttccacattccaaaccctaagagatattcttattgcgggaaaaacaacaaggctcccttgcccttgctgagttttcatacactgctacaaaagcagaactatttagaaggagttaaaatgtcttttgtaaagtttcttacacagctttctttttttcccttttttacaggcaacttcgggctcgctggagaacaactaaggggcaccaaagccctctgaggttttactttaaggttcgctgtatgtttgccttggacaaaaaggctacctaccacgtgctatccagtaatatacttaaataagccaatacttagatctactgtaaggcagatgctaattataaggcattaagtaagcaaatagtgccctcagctactgcagaagaaaagtcccactgaggaaaagaaagtcttgtgatttttaaaggcaagttttcaagtgctctcatagttctatcctctaattccattaaatccatactaggagcgtcagtgagggttttcatagcttttggaaatactttggtctctgaactgtaattagcgcaagaagtaaaaacagaaacgtcaaacgtcaaatgtttgctttgttacctggaggactaaatgtagatgtctttagtatactttgtatgttcttaaatattggaagataattttgtgaatctgtagattttattttttcagtcttaccttacaaatttcttttctatgaataatagaggaactcacggcactctgccacttgttaatgaaaggaagtgcagaggatttagaaaagtacatgatccccagaccacaacaaaccaaaacataaactcatgtctgtgtcccatggtcatagtcaaagattttgtactgctaaaattaccaaataatttaaataaagtggatttgaacacaatttgaaggtgtctttctgattaacatgatagaaacttcacataaatcagtttcttagatctagatatacaaaagcactgtgacaaatgg

SEQ ID NO: 4 - DNA, 418 bp, Homo sapiens
gatttgtgtgtgcaccgcgtctccagcgatcccggatccactgcgctgccaggggcctgggggtgggtctcttgctgtctctgcgacgacatccttacgtttcggcactctaatgctgggtttgtgcgtgtgtgtctgcttagcggtctagcgggctgttaggcctccctcgcccccagctccttggtcgctcagctcctccaccgcagcccagcagtgagacgcgcgcgcagccagctccccacgagatggaacagaccgaagtgctgaagccacggaccctggctgatctgatccgcatcctgcaccagctctttgccggcgatgaggtcaatgtagaggaggtgcaggccatcatggaagcctacgagagcgaccccatcgagtgggcaatgtacgccaagttcgaccagtacag

SEQ ID NO: 5 - DNA, 78 bp, Homo sapiens
gtatacccgaaatcttgtggatcaaggaaatggaaaatttaatctgatgattctctgttggggtgaaggacatggcag

SEQ ID NO: 6 - DNA, 155 bp, Homo sapiens
cagtattcatgatcataccaactcccactgctttctgaagatgctacagggaaatctaaaggagacattatttgcctggcctgacaaaaaatccaatgagatggtcaagaagtctgaaagagtcttgagggaaaaccagtgtgcctacatcaatg

SEQ ID NO: 7 - DNA, 170 bp, Homo sapiens
attccattggcttacatcgagtagagaacatcagccatacggaacctgctgtgagccttcacttgtacagtccaccttttgatacatgccatgcctttgatcaaagaacaggacataaaaacaaagtcacaatgacattccatagtaaatttggaatcagaactccaaat

SEQ ID NO: 8 - DNA, 735 bp, Homo sapiens
gcaacttcgggctcgctggagaacaactaaggggcaccaaagccctctgaggttttactttaaggttcgctgtatgtttgccttggacaaaaaggctacctaccacgtgctatccagtaatatacttaaataagccaatacttagatctactgtaaggcagatgctaattataaggcattaagtaagcaaatagtgccctcagctactgcagaagaaaagtcccactgaggaaaagaaagtcttgtgatttttaaaggcaagttttcaagtgctctcatagttctatcctctaattccattaaatccatactaggagcgtcagtgagggttttcatagcttttggaaatactttggtctctgaactgtaattagcgcaagaagtaaaaacagaaacgtcaaacgtcaaatgtttgctttgttacctggaggactaaatgtagatgtctttagtatactttgtatgttcttaaatattggaagataattttgtgaatctgtagattttattttttcagtcttaccttacaaatttcttttctatgaataatagaggaactcacggcactctgccacttgttaatgaaaggaagtgcagaggatttagaaaagtacatgatccccagaccacaacaaaccaaaacataaactcatgtctgtgtcccatggtcatagtcaaagattttgtactgctaaaattaccaaataatttaaataaagtggatttgaacacaat

SEQ ID NO: 9 - DNA, 20 bp, Artificial
  Oligonucleotide PRIMER - exon 1 forwards primer
gagggccgttggtacattcc

SEQ ID NO: 10 - DNA, 19 bp, Artificial
  Oligonucleotide primer - exon 1 reverse primer
gccagtcactttgggctgc

SEQ ID NO: 11 - DNA, 21 bp, Artificial
  Oligonucleotide primer - exon 2 forwards primer
gcttgtttcagcaacgaactt

SEQ ID NO: 12 - DNA, 25 bp, Artificial
  Oligonucleotide primer - exon 2 reverse primer
ttttcatatgctagaaaacatgctc

SEQ ID NO: 13 - DNA, 22 bp, Artificial
  Oligonucleotide primer - exon 3 forwards primer
gaccatctactgagttcagttg

SEQ ID NO: 14 - DNA, 20 bp, Artificial
  Oligonucleotide primer - exon 3 reverse primer
tcttcccacttgcccttaga

SEQ ID NO: 15 - DNA, 20 bp, Artificial
  Oligonucleotide primer - exon 4 forwards primer
tttttccttcccggatattg

SEQ ID NO: 16 - DNA, 20 bp, Artificial
  Oligonucleotide primer - exon 4 reverse primer
cagtgccaacctacagagca

SEQ ID NO: 17 - DNA, 20 bp, Artificial
  Oligonucleotide primer - exon 1 alternative reverse primer
gaggtcagcaaggccagtag

(19) United States
(12) Patent Application Publication
(10) Pub. No.: US 2009/0297475 A1
(43) Pub. Date: Dec. 3, 2009

(54) CYSTEINE DIOXYGENASE POLYMORPHISMS

(75) Inventors: Rosemary Hope Waring, Birmingham (GB); David Boyer Ramsden, Worcestershire (GB); Lucy Jane Wilkinson, Worcestershire (GB); Hugh Kikuchi, London (GB)

Correspondence Address: RATNERPRESTIA, P.O. BOX 980, VALLEY FORGE, PA, 19482, US

(73) Assignee: THE UNIVERSITY OF BIRMINGHAM, Birmingham, West Midlands (UK)

(21) Appl. No.: 11/996,281

(22) PCT Filed: Jul. 20, 2006
(86) PCT No.: PCT/GB2006/002698

§ 371 Date: Sep. 29, 2008

(30) Foreign Application Priority Data
Jul. 20, 2005 (GB) 0514913.3

(51) Int. Cl. A61K 38/21; C12Q 1/68; C07H 21/04; A61P 19/02; C07K 16/00

(52) U.S. Cl. 424/85.5; 435/6; 536/24.31; 536/23.5; 530/389.1

(57) ABSTRACT

The invention relates to the detection and identification of polymorphisms in cysteine dioxygenase (CDO) for the use of that diagnosis to identify a propensity in a patient towards rheumatoid arthritis and/or to have side effects with a number of drugs, to nucleic acid and isolated proteins encoding the polymorphisms, to assays for CDO activity and for the identification of compounds affecting CDO activity, and additionally to use of interferon-γ optionally in combination with different compounds, to treat rheumatoid arthritis.

### CROSS REFERENCE TO RELATED APPLICATIONS

This application is the U.S. national phase of PCT Application No. PCT/GB2006/002698, filed Jul. 20, 2006, and claims priority to (Great Britain Application No. 0514913.3, filed Jul. 20, 2005, the contents of each of which are incorporated by reference herein, in their entirety.

### FIELD OF THE INVENTION

The invention relates to the detection and identification of polymorphisms in cysteine dioxygenase (CDO) for the use of that diagnosis to identify a propensity in a patient towards rheumatoid arthritis and/or to have side effects with a number of drugs, to nucleic acid and isolated proteins encoding the polymorphisms, to assays for CDO activity and for the identification of compounds affecting CDO activity, and additionally to use of interferon-γ optionally in combination with different compounds, to treat rheumatoid arthritis.

### BACKGROUND OF THE INVENTION

Rheumatoid Arthritis (RA) is an autoimmune disease that is characterised by chronic inflammation and tissue destruction in the joints of a sufferer. The exact reasons for how this disease is initiated are unknown. However, there is evidence of a genetic susceptibility and microbial transmission, but neither have been satisfactorily characterised. At the onset of a disease, there is an increased release of proinflammatory cytokines resulting in chronic inflammation around the limb joints, slowly contributing to the increasing disability of the sufferer. The synovial membrane around the joints grows to a far larger than normal size. The proliferation of this tissue leads to the formation of the pannus, a mass of fibroblast cells that cause ligament and bone destruction as it spreads. Often the joints inflame, as synovial fluid collects around them. The fluid itself is a mixture of body serum, lymphocytes and numerous secreted chemical signals. Amongst the chemical signals are proinflammatory cytokines that perpetuate the chronic inflammation and progressive incapacity of the affected individual.

In the context of rheumatoid arthritis, two examples of proinflammatory cytokines are TNFα (Tumor Necrosis Factor alpha) and IL-1 (interleukin-1). Hyper-expression of signalling factors in the early stages of the onset of rheumatoid arthritis is known to contribute greatly to the development of the disease symptoms, and the perpetuated synthesis of the cytokines contributes to the maintenance of the diseased state. Blocking the cytokines has been the basis of much research into treatment of rheumatoid arthritis. For example, there are a number of anti-TNF drugs on the market (such as Ifliximab, produced by Centocor and Etanercept, produced by Amgen). Additionally, anti-IL-1 drugs are also known (such as Anakira, produced by Amgen).

Cysteine dioxygenase (CDO) is an enzyme that is involved in the metabolism of sulphur-containing compounds. It catalyses the formation of cysteine sulphonic acid from cysteine. This in turn is utilised in a number of different pathways, for example to produce taurine, cysteic acid, alanine and β-sulphinylpyruvate. The latter compound is of particular interest because it is metabolised to sulphite and then to sulphate. Whilst other pathways for sulphate formation exist, they are of much less importance. Various conditions with an autoimmune or inflammatory component, such as rheumatoid arthritis, systemic lupus erythematosus, primary biliary cirrhosis, Crohn's disease and ankylosing spondylitis have been shown to be linked with high plasma levels of cysteine and low plasma concentrations of inorganic sulphate in patients with these diseases.

Sulphate is involved in the sulphonation of the synovial fluid, proteins and glycosaminoglycans found in the joint itself. Decreasing the level of sulphonation causes a decrease in the protective and lubricating properties of these, thus exacerbating the disease condition.

Wilkinson L. J. and Waring R. H. (Toxicol in vitro (2002), pages 481-483) demonstrated that CDO is a rate-limiting enzyme involved in the oxidative degradation of inorganic sulphate. They noted that inflammatory conditions, such as rheumatoid arthritis (RA) have been linked to high cysteine:sulphate ratios in patients. They showed tumour necrosis factor-α (TNF-α and transforming growth factor-β (TGF-β) were able to inhibit the expression of CDO production in some cell lines. They concluded that such cytokines may be involved in the worsening of RA by lowering CDO expression. In this article neuronal and hepatic (Chang) human cell lines were assayed for CDO by detection of the CDO using antibodies and western blotting.

Current tests for rheumatoid arthritis include the rheumatoid factor test. Rheumatoid factor is an antibody that is eventually present in the blood of most RA patients. However, not all RA patients test positive for RF, especially early in the disease which makes the negative result of limited value. Conversely, other patients who test positive for RF never develop the disease. This test is positive for 60-70% of patients with rheumatoid arthritis and this is usually an argument for diagnosing rheumatoid arthritis in patients with arthritis and no other obvious diagnosis. However, rheumatoid factor also occurs in other diseases.

There are also a number of general biochemical indicators, which give some impression of the activity and extent of inflammation, but which are again not specific for rheumatoid arthritis. Radiological examination using X-rays or MRI may also detect rheumatoid arthritis, but again later in the progression of the disease. Additionally, HLA-typing may be used looking for markers associated with DR1 and DR4. 70-90% of patients with rheumatoid arthritis in hospital-based surveys have these markers, but some 30% of the general population also share these markers. Hence, there is a need for further diagnostic tests for rheumatoid arthritis.

### BRIEF DESCRIPTION OF THE DRAWINGS

FIG. 1 shows the intron and exon arrangement of the human CDO gene.

FIG. 2 shows the intron and exon sequences of exons 1, 2, 3 and 4 of CDO. The capitalised lettering indicates the exon sequence, with a lower black case text indicating the intron sequence. Numbers of the lines are the positions of the first base of that line of code. The CDO sequence was obtained from the National Center for Biotechnology Information (www.ncbi.nlm.nih.gov). The accession number for CDO is U60232 (U60232.1, GI:2138108).

FIG. 3 shows a diagram showing the first sets of primers designed for the amplification of CDO exon 2 (part A), exon 3 (part B), exon 4 (part C) and exon 1 (part D). Primer regions are shown in lower case and are not in bold and are underlined, adjacent to arrows indicating the direction of the synthesis from the primers. Two different exon 1 reverse primers are shown. There is a single nucleotide difference between the alternative exon 1 reverse primer (SEQ ID No 17) and the primer binding (i.e. target) region of exon 1.

FIG. 4 shows a gel image of touchdown PCR products. Lanes (counting from left of image): 1 control exon 4 (2) control exon 3 (3) control exon 2 (4) DNA ladder.

FIG. 5 shows a gel image of PCR products using a control template with the addition of DMSO in the reactions. Exons 2, 3 and 4 can be seen (circled). DMSO was not used during PCR amplification of exon 1 (not shown).

FIG. 6 shows:

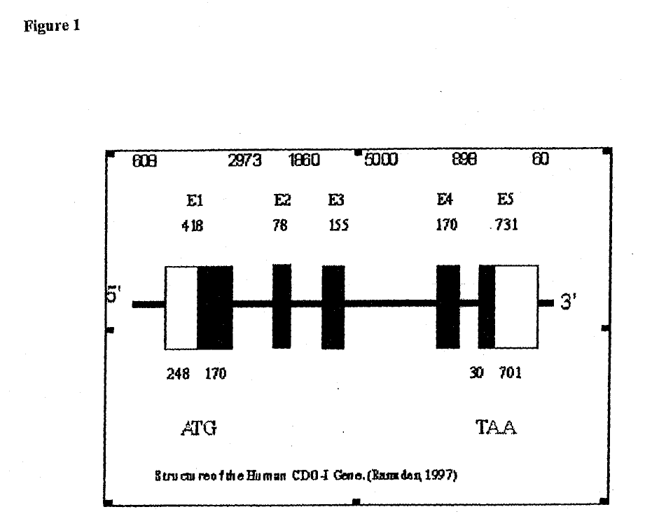

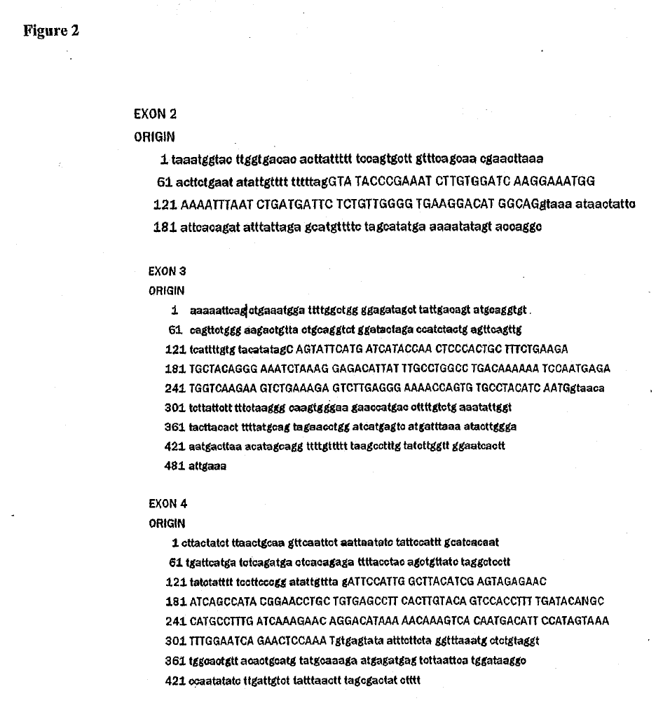

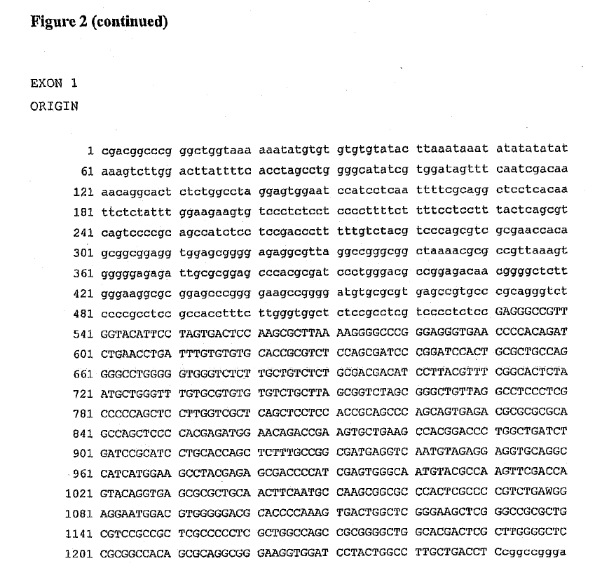

A) Magnesium gradient series of control PCR reactions. Lanes (counting from left) (1) E4, [MgCl₂] 4; (2) E3, [MgCl₂] 4; (3) E2, [MgCl₂] 4; (4) E4, [MgCl₂] 3; (5) E3, [MgCl₂] 3; (6) E2, [MgCl₂] 2; (7) E4, [MgCl₂] 2; (8) E3, [MgCl₂] 2; (9) E2, [MgCl₂] 2; (10) LADDER.

B) Magnesium gradient series of Rheumatoid Arthritis template PCR reactions (including DMSO). Lanes: (1) E4, [MgCl₂] 4; (2) E3, [MgCl₂] 4; (3) E2, [MgCl₂] 4; (4) E4, [MgCl₂] 3; (5) E3, [MgCl₂] 3; (6) E2, [MgCl₂] 3; (7) E4, [MgCl₂] 2; (8) E3, [MgCl₂] 2; (9) E2, [MgCl₂] 2; (10) E4, [MgCl₂] 1; (11) E3 [MgCl_{2] 1}; (12) E2, [MgCl₂] 1; (13) LADDER.

In these figures, exons are identified by E2 (exon 2), E3 (exon 3) and E4 (exon 4) and the volume of MgCl₂ used is shown in microlitres. For example. Lane (1) shows Exon 4 with which was amplified in the presence of 4 μl MgCl₂.

FIG. 7 shows Gel image of PCR products from Touchdown PCR. All exons from both control and RA DNA have been successfully amplified.

FIG. 8 shows, by way of example, comparison of two sequencing results, both from RA exon 3. (A) PCR product at purification result, poor sequence, dual signal, many “N”s (undefined nucleotides) (B) Gel extraction result, good sequence, and well-defined base positions.

FIG. 9 shows predicted effects of polymorphisms on amino acid substitutions. All potential SNPs (see Table 3) have been included, therefore the affects illustrated are more pronounced that they would occur in vivo. Loci marked with bold arrows indicate a substitution SNP. Lighter arrows indicate an addition SNP. The underlining indicates amino acid sequence that differs from normal (control) sequence. (A) Exon 2 polymorphisms (B) Exon 3 polymorphisms (C) Exon 4 polymorphisms.

FIG. 10 shows filtered SNP analysis. Exon 3 shows greatly reduced change in the amino acid sequence, whilst Exon 4 maintains a very high level of amino acid disruption, despite the reduced number of SNPs.

FIG. 11 shows a gel image of PCR products of Exon 1. Lane 1: DNA ladder, Lanes 2-7: exon 1, Lane 8: positive control.

### DETAILED DESCRIPTION OF THE INVENTION

The inventors have unexpectedly found that there are a number of polymorphisms found in the DNA encoding CDO or the regulatory regions controlling the expression of CDO in patients with RA and people with a family history of RA, thus suggesting a link between RA and low concentrations of CDO. Furthermore, they have found new assays for CDO expression and have identified that a number of drugs, including Interferon-γ (IFN-γ) increase CDO expression, thus showing that IFN-γ may be used to treat RA.

EP 0974358A suggests using IFN-γ to treat HTLV-1-related diseases. HTLV-1 is associated with adult T-cell leukemia. It also speculates that it may be involved in a number of other diseases, including Sjögren's syndrome, systemic lupus erythematosus, uveitis and chronic rheumatoid arthritis. It is now known that HTLV is only rarely, if ever, associated with chronic RA.

The inventors of the current application considered that CDO may be a key limiting step in the production of sulphate for use in the sulphonation of the components of the joints. They therefore studied nucleic acid samples of two different populations: a population having rheumatoid arthritis and a controlled population. They unexpectedly found that there were a high level of mutations in the rheumatoid arthritis population compared to the controlled population when the CDO gene was sequenced. Furthermore, they also noted that one of the controls who had one of the mutations identified was only aged 25 but had a family history of rheumatoid arthritis, thus suggesting that the mutations are not only markers suggesting the presence of rheumatoid arthritis, but also are markers suggesting the potential development of rheumatoid arthritis later in a person's life span.

Accordingly, the invention provides a method for the diagnosis of a CDO-mediated condition, which method comprises:
(i) obtaining a sample from an individual;
(ii) detecting the presence or absence of a variant CDO protein, or a nucleic acid encoding a CDO variant and/or a variant of a CDO regulatory region;
(iii) determining the status of the individual by reference to polymorphism in the CDO gene and/or its regulatory region.

The decrease in the amount of sulphonation that occurs may be due to either decreased expression of CDO protein. Preferably the regulatory region is 5′ to the region encoding CDO. It may be a promoter or enhancer region. Alternatively, CDO protein may be produced, but the mutation in the DNA encoding the protein results in a change in one or more amino acids in the CDO, thus resulting in CDO with a lower enzymatic activity. Both the reduced level of CDO protein production and the reduced enzymatic activity of CDO would produce a reduction in the amount of sulphate produced. Hence, the variant CDO may be detected by either looking for polymorphisms within the nucleic acid encoding the CDO variant, its regulatory region or alternatively looking for changes in the protein structure of the protein produced from such variants compared with normal individuals having normal levels of CDO.

Normal levels of CDO are thought to be in the range of 10-30, possibly 5-100, picomoles per mg of protein. It is though that the enzymatic activity of normal CDO is in the range of 10-30, possibly 5-100, picomoles per mg of protein per hour.

The invention also provides methods for diagnosis of one or more polymorphisms in the CDO gene, or its regulatory sequence in a human comprising determining the sequence of a nucleic acid molecule encoding at least a portion of the CDO gene and/or a CDO regulatory region and determining the status of the human by reference to polymorphism in the CDO gene and/or its regulatory region.

Polymorphism refers to the occurrence of two or more genetically determined alternative alleles or sequences within a population. A polymorphic marker is the site at which divergence occurs. For example, a base, such as adenine may be replaced by a different base such as thymine. Alternatively, one or more additional bases may be inserted at the site. Polymorphisms where a base is substituted for another base may result in, for example, a change in a codon so that a different amino acid is inserted into the protein when messenger RNA derived from the nucleic acid sequence is translated into the final enzyme. For example, GCC is a codon encoding alanine. If this codon is changed so that it contains an adenine residue, such as GAC, the codon will be misread and will result in the insertion of an aspartic acid residue instead of alanine. Substitution of one or more bases may also result in, for example, the production of stop codons resulting in termination of the formation of the protein. Such substitutions, if they occur in the regulatory sequences, such as the promoter sequences upstream of the CDO gene, may result in the reduction in the levels of expression of the CDO. Alternatively, a change in a sequence encoding a processing signal may also affect expression of the final CDO product.

Other mutations include addition mutations where one or more bases are inserted into the DNA and deletion mutations where one or more bases are deleted from the DNA encoding the regulatory sequence or the CDO sequence itself. Such deletions may result in the production of truncated proteins, or alternatively result in a decrease in the level of expression of the CDO gene.

Preferably, two or more polymorphisms are looked for. Preferably, the markers have at least two alleles, each occurring at a frequency of greater than 1%, more preferably at least 10%, 15%, 20%, 30% or more of a selected population, such as a population having RA.

Single nucleotide polymorphisms (SNPs) are generally, as the name implies, single nucleotide or point variations that exist in the nucleic acid sequence of some members of a species. Such polymorphism variation within the species are generally regarded to be the result of spontaneous mutation throughout evolution.

Polymorphisms may also affect mRNA synthesis, maturation, transportation and stability. Polymorphisms which do not result in amino acid changes (silent polymorphisms) or which do not alter any known consensus sequences, may nevertheless have a biological effect, for example by altering mRNA folding, stability, splicing, transcription rate, translation rate or fidelity. Indeed, it has been reported that even polymorphisms that do not result in an amino acid change can cause different structural folds of mRNA with potentially different biological functions (Shen, et al. (1999) PNAS USA, Vol. 96, pages 7871-7876). Thus, changes that occur outside the coding region, such as in the intron sequences and promoter regions may affect the transcription and/or messenger stability of the sequences and thus affect the level of the CDO protein in cells.

Preferably the polymorphism is selected from:

| Exon | Position | SNP | Type |
|---|---|---|---|
| Exon 1: | +673 | A | insertion |
| Exon 1: | +697 | G | insertion |
| Exon 1: | +1103 | C-A | substitution |
| Exon 3: | +15 | A-T | substitution |
| Exon 3: | +16 | T-C | substitution |
| Exon 3: | +17 | A-C | substitution |
| Exon 3: | +29 | T-A | substitution |
| Exon 3: | +30 | G | addition |
| Exon 4: | +33 | A-T | substitution |
| Exon 4: | +34 | G-C | substitution |
| Exon 4: | +43 | A | addition |
| Exon 4: | +46 | C-A | substitution |

CDO is known to comprise five exons. The nucleotide sequence of CDO is available through Genbank which can be accessed at http://www.ncbi.nlm.nih.gov. The nucleotide sequence is available in three segments. CDO Segment 1 is 4186 nucleotides long and includes exon 1 (nucleotides 609-1026) and exon 2 (nucleotides 4000-4077) and can be accessed via Genbank Accession Number U60232.1, GI:2138108. CDO Segment 2 is 2739 nucleotides long and includes exon 3 (nucleotides 1735-1889) and can be accessed via Genbank Accession Number U78979.1, GI: 1699035. CDO Segment 3 is 2995 nucleotides long and includes exon 4 (nucleotides 1115-1284) and exon 5 (nucleotides 2173-2907) and can be accessed via Genbank Accession Number U80055.1, GI:2138109.

In the sequence listing, Segment 1=SEQ ID No. 1, Segment 2=SEQ ID No. 2, Segment 3=SEQ ID No. 3, Exon 1=SEQ ID No. 4, Exon 2=SEQ ID No. 5, Exon 3=SEQ ID No. 6, Exon 4=SEQ ID No. 7 and Exon 5=SEQ ID No. 8.

The SNP co-ordinates, given above, refer to the wild-type CDO nucleotide sequence available via the NCBI databank. For Exon 1, the co-ordinates correspond to the numbering of Segment 1 (i.e. Seq. ID No. 1). Therefore, the polymorphisms identified are not located within the coding sequence of Exon 1 and instead lie within the flanking regions of Exon 1. For Exons 3 and 4, the SNP co-ordinates correspond to the numbering of Exons 3 (i.e. Seq. ID No. 6) and 4 (i.e. Seq. ID No. 7), respectively.

The polymorphism may be detected in genomic DNA. Alternatively, it may be detected in RNA, such as mRNA or cDNA obtained from such mRNA.

The methods of the invention provide an indication that the human has decreased CDO concentrations and/or expresses CDO having decreased enzymatic activity.

The sample may be obtained from, for example, blood or other body tissues, or a mouth check swab. The diagnosis is preferably carried out in vitro.

The CDO-mediated condition may be rheumatoid arthritis and/or the development of side-effects with one or more drugs selected from D-penicillamine and gold thiomalate.

The presence or absence of one or more polymorphisms is used to assist in the diagnosis.

The ability to identify, for example the propensity of an individual or human to develop rheumatoid arthritis allows individuals to be treated with, for example, methotrexate prior to the formation of the painful symptoms of rheumatoid arthritis. This will help prevent the patient developing erosive rheumatoid arthritis. It means that such individuals can be identified early and their quality of life can be considerably improved by helping to prevent the full onset of rheumatoid arthritis.

Drugs such as D-penicillamine and gold thiomalate are known to cause side-effects in rheumatoid arthritis patients being treated with such compounds. The drugs cannot be properly oxidized because, it is thought, due to the low levels of CDO or low levels of active CDO in those patients. Hence, being able to identify those patients which are likely to get such side-effects will assist because it allows the patients to be placed on alternative medication.

Methods for identifying the presence or absence of a polymorphism within a region of nucleic acid are in themselves generally known.

Preferably, the polymorphism is amplified by polymerase chain reaction prior to identifying the polymorphism. This may simply be achieved by using a primer directed towards a sequence upstream of the polymorphism, amplifying a stretch of nucleic acid encoding the polymorphism and then sequencing the amplified sequence. Alternatively, the primer may be directed to the site of the polymorphism with the sequence of the primer substantially complementary to the polymorphism sequence of interest so that the primer hybridises to the polymorphism site and is then used to produce a PCR product. If the polymorphism of interest is not there, then the primer will not bind to that site and will not produce a PCR product. Consequently there will be no PCR product available for detection. Real-time PCR may be used to identify SNPs. Real-time PCR has evolved because of the advent of various chemistries that label primers, probes and amplicons with, for example, fluorogenic molecules. Reactions are usually set up in capillary tubes that act as cuvettes, allowing the fluorescent signal to be monitored at the end of each cycle. This allows target amplification and detection to occur in the same tube, creating a closed tube system that eliminates the need of post-amplification processing and detection. For example, WO97/46712 discloses thermal cycling methods and devices.

WO 00/18965 discloses methods directed to the detection of the presence of mutations or polymorphisms. This uses the polymerase chain reaction and fluorescently labelled oligonucleotide hybridisation probes to identify mutations and polymorphisms based on melting curve analysis of the hybridisation probes. The fluorophores used may be one of a number of different compounds known in the art, including ethidium bromide, YO-PRO-1 and SYBR Green I. Fluorescent resonance energy transfer (FRET) relies on the adjacent annealing of two hybridisation probes (Lay N. J. and Wittwer C. T. Clin. Chem. (1997), Vol. 43, pages 2262-2267). The first probe contains a donor dye at its 3′ end, and the other contains an accepted dye at its 5′ end. When light is added through an external source, the donor dye is excited and transfers energy to the acceptable dye in the fluorescent resonance energy transfer process. Only when both primers anneal in close proximity, is energy transfer possible. Hence, such techniques allow the detection of the binding of complementary primers to specific polymorphisms.

Alternatively, less complex techniques, such as amplification of the nucleic acid around a potential polymorphism site may be undertaken. The amplified DNA may be separated, for example, by using electrophoresis and the DNA may be probed using a labelled probe complementary to the polymorphism of interest. The labelled probe may be labelled using radioactive labels or, for example, fluorescent labels known in the art.

The method used preferably includes a method according to the invention in which a nucleic acid sequence is determined by a method selected from ARMS-allele specific amplification, allele specific hybridisation, oligonucleotide ligation assay and restriction fragment length polymorphism (RFLP). ARMS-allele specific amplification is described in, for example, EP 0332435 and U.S. Pat. No. 5,595,890. This relies on the complementarity of the 3′ terminal nucleotide of the primer and its template. The 3′ terminal nucleotide of the primer being either complementary or non-complementary to the specific mutation, allele or polymorphism to be detected. RFLP relies on the altering of the sequence affecting a restriction endonuclease site, thus preventing digestion of that site by a restriction enzyme.

The presence or absence of one or more, preferably 2 or more, different polymorphisms in an individual allows the propensity of a person to develop, for example, rheumatoid arthritis and/or develop side-effects to drugs to be determined. Other diseases which may be associated with this CDO defect include autoimmune diseases such as Systemic Lupus Erythrematosus, Sjögren's syndrome, uveitis, Chrohn's disease, ankylosing spondylitis, neurogenerative diseases such as Parkinson's disease and Alzheimer's disease and Motor Neurone Disease. Further diseases which may be associated with this CDO defect include those involving antibodies to inflammatory cytokines.

The invention also provides allele specific probes or primers for use in the method according to the invention. As indicated above, the processes used to identify polymorphisms are themselves generally known. The identification of suitable probes or primers, once the polymorphism site has been identified, will itself normally be within the knowledge of the skilled person.

Preferred probes or primers comprise the following sequences:

[SEQ ID No. 9]

Exon 1: Primer fwd: gagggccgttggtacattcc

[SEQ ID No. 10]

Exon 1: Primer rev: gccagtcactttgggctgc

[SEQ ID No. 17]

Exon 1: Primer rev: ctactggccttgctgacctc

[SEQ ID No. 11]

Exon 2: Primer fwd: gcttgtttcagcaacgaactt

[SEQ ID No. 12]

Exon 2: Primer rev: ttttcatatgctagaaaacatgctc

[SEQ ID No. 13]

Exon 3: Primer fwd: gaccatctactgagttcagttg

[SEQ ID No. 14]

Exon 3: Primer rev: tcttcccacttgcccttaga

[SEQ ID No. 15]

Exon 4: Primer fwd: tttttccttcccggatattg

[SEQ ID No. 16]

Exon 4: Primer rev: cagtgccaacctacagagca

Exon 1 primer were initially designed to hybridise to the sequence of accession no D85778.1 GI: 1747325 (exon 1) which is publicly available at www.ncbi.nlm.nih.gov.

Preferred probes specifically hybridise to the region of nucleic acid containing the SNP of interest and the skilled person is familiar with techniques for designing primers with suitable characteristics, e.g. specificity, melting temperature.

Preferably, the probes or primers are capable of detecting the following polymorphisms:

| Exon | Position | SNP | Type |
|---|---|---|---|
| Exon 1: | +673 | A | insertion |
| Exon 1: | +697 | G | insertion |
| Exon 1: | +1103 | C-A | substitution |
| Exon 3: | +15 | A-T | substitution |
| Exon 3: | +16 | T-C | substitution |
| Exon 3: | +17 | A-C | substitution |
| Exon 3: | +29 | T-A | substitution |
| Exon 3: | +30 | G | addition |
| Exon 4: | +33 | A-T | substitution |
| Exon 4: | +34 | G-C | substitution |
| Exon 4: | +43 | A | addition |
| Exon 4: | +46 | C-A | substitution |

The probes or primer of the invention preferably correspond to, that is, they are complementary to the polymorphism to be detected. The probes or primers of the invention are preferably 17-50 nucleotides, more preferably about 17-30 nucleotides in length. The primer or probe may correspond to about 6-8 of the nucleotides of the polymorphism.

If the probe or primer is simply being used as a probe, that is as a label, rather than as a primer for PCR, the size of the probe may be reduced, for example to 8-25, preferably 8-15 bases in length.

Preferably, the probes or primers of the invention have at least 90, 91, 92, 93, 94, 95, 96, 97, 98 or 99% homology to the polymorphism site. Preferably they are identical to the polymorphism site, or alternatively complementary to the polymorphism site. The probes or primers may, of course, bind to the strand of nucleic acid encoding the CDO or its regulatory region containing the polymorphism, or alternatively bind to its complementary strand where present.

Diagnostic kits comprising one or more probes or primers according to the invention are also provided.

A further aspect of the invention provides an isolated nucleic acid of at least 5 bases long encoding a CDO or a CDO-regulatory polymorphism or complementary to such a polymorphism. Preferably the polymorphism is selected from:

| Exon | Position | SNP | Type |
|---|---|---|---|
| Exon 1: | +673 | A | insertion |
| Exon 1: | +697 | G | insertion |
| Exon 1: | +1103 | C-A | substitution |
| Exon 3: | +15 | A-T | substitution |
| Exon 3: | +16 | T-C | substitution |
| Exon 3: | +17 | A-C | substitution |
| Exon 3: | +29 | T-A | substitution |
| Exon 3: | +30 | G | addition |
| Exon 4: | +33 | A-T | substitution |
| Exon 4: | +34 | G-C | substitution |
| Exon 4: | +43 | A | addition |
| Exon 4: | +46 | C-A | substitution |

and sequences complementary to such sequences. Preferably, the isolated nucleic acid comprises at least 10, 15, 20, 30 or 40 bases.

More preferably, the isolated nucleic acid molecule encodes a CDO or a fragment thereof, which more preferably contains a polymorphism.

Isolated CDO protein obtainable from such nucleic acid molecules are also provided. Methods of producing recombinant proteins from isolated nucleic acid molecules are well-known in the art. The isolated nucleic acid molecule may be cloned into a suitable vector and expressed in a suitable prokaryotic or eukaryotic host.

Accordingly, vectors and host cells containing the isolated nucleic acid molecules of the invention are also provided by the invention. Such vectors preferably contain a suitable promoter and termination sequences of the type known in the art. Alternatively, the CDO's own promoter and regulatory sequences may be used, thus allowing the effect of a polymorphism on one or more of these sequences to be studied. In the latter case, the regulatory sequence may be attached to a suitable reporter sequence known in the art, such as β-galactosidase or green fluorescent protein, the expression of which can be easily assayed.

As indicated above, a polymorphism may result in a change in the amino acid sequence of the CDO, thus resulting in change in the structure of the enzyme. Such changes in the structure of the enzyme may be readily identified by the isolation of one or more antibodies, such as monoclonal antibodies specific for the CDO protein. That is, the antibodies do not also bind CDO not containing the polymorphism in the encoding nucleic acid sequence. The production of monoclonal antibodies is itself well know, for example via the methods of Kohler and Milstein.

A further aspect of the invention provides a method of determining the propensity of an individual to a CDO-mediated condition, comprising:

(i) obtaining a sample from the individual; and

(ii) detecting the presence of a CDO containing the polymorphism using an antibody according to the invention.

The inventors unexpectedly have found that CDO is expressed in white blood cells. Previously, CDO has been found in brain and lung tissue. Such tissues are not readily accessible. Accordingly, preferably the sample used in a method of the invention, for example of the variant CDO or nucleic acid encoding the CDO, or indeed its regulatory sequence, is obtained from white blood cells.

The relatively ready availability of tissue samples containing CDO allows an assay for CDO activity to be produced. Accordingly a further aspect of the invention provides a method of determining the effect of a compound on CDO activity comprising:

(i) Providing a sample of white blood cells;

(ii) Contacting the white blood cells with a CDO substrate and the compound; and

(iii) Determining the effect of the compound on the conversion of the substrate to a detectable product by CDO in the white blood cells.

Examples of CDO substrates include cysteine, S-carboxy methyl cysteine, S-methyl cysteine, auro thiomalate, N-acetyl cysteine, alkyl-cysteines and D-penicillamine. These substrates breakdown to generate products including cysteine sulphinic acid. Such methods may be used to identify a drug candidate for the treatment of rheumatoid arthritis. That is, the effect of, for example, a cytokine or alternatively a different compound, on CDO expression within white blood cells may be determined.

Further work by the inventors has indicated that interferon-γ (IFN-γ) can increase CDO expression. This is in contrast to TNF-γ which decreases CDO expression.

As CDO is pivotal to the uptake of sulphate in the human body and low levels of sulphate are associated with rheumatoid arthritis, it is expected that IFN-γ may be used as a therapeutic agent.

The invention therefore provides a method of treating rheumatoid arthritis comprising administering a pharmaceutically effective amount of interferon-γ (IFN-γ). Preferably, the rheumatoid arthritis is non-HTLV-1 associated rheumatoid arthritis. As already indicated, HTLV-1 associated rheumatoid arthritis is thought to be relatively rare.

The interferon-γ may be administered in combination with one or more compounds that are capable of raising the level of sulphate production. These include: cysteine, methionine, D-penicillamine, N-acetyl cysteine, S-alkyl cysteine, γ-glutamylcysteine, interleukin-4 and interleukin-10.

IFN-γ for use in the manufacture of medicament to treat rheumatoid arthritis, optionally with the sulphate inducing compounds listed above, is also provided.

A still further aspect of the invention provides a pharmaceutically acceptable composition comprising interferon-γ in combination with a sulphate inducing compound such as one or more of: cysteine, methionine, D-penicillamine, N-acetyl cysteine, S-alkyl cysteines, γ-glutamylcysteine, interleukin-4, S-carboxy methyl, S-methyl cysteine and interleukin-10.

Interferon-γ is already licensed for clinical use in the treatment of chronic granulomatous disease to help reducing serious infections. That dosage is 90 micrograms three times a week. The doses suitable for the treatment of rheumatoid arthritis are expected to be within similar dosage limits.

The IFN-γ and optionally the other compounds referred to above, may be used in combination with one or more stabilisers, such as glucose, sucrose or trehalose. A pharmaceutically acceptable salt of IFN-γ and/or the sulphate inducing compounds may be used.

Additionally one or more acceptable pharmaceutically acceptable diluents, excipients, fillers, stabilisers, pH-controlling agents, biologically-active substances, etc. may be incorporated.

Preferably, the material is formed into an orally-administrable agent, such as a capsule, powder or tablet. Alternatively, it may be used via another route, such as intramuscular injection.

The invention will now be described by way of example, with reference to the figures. The examples are intended to illustrate and not to limit the invention.

### EXAMPLE 1

### Materials and Methods

Genomic DNA samples were obtained from blood from rheumatoid arthritis (RA) patients and control volunteers (C). Blood was collected into EDTA treated tubes by venopuncture. PCR was carried out as described below.

Primer Design. Specific PCR primers were designed for amplification of exons 1, 2, 3 and 4 of CDO. Primers were designed using sequence data obtained from the NCBI databases (www.ncbi.nlm.nih.gov) and the Primer 3 internet-based primer designing tool (http://frodo.wi.mit.edu/cgi-bin/primer3/primer3 www.cgi which is hosted by the Massachusetts Institute of Technology, http://web.mit.edu/index.html). Oligonucleotide primers were synthesised by Alta Biosciences (Birmingham, UK) and subsequently diluted to 100 p.mole μl⁻¹ concentration using distilled water.

Polymerase Chain Reaction (PCR). CDO exons 2, 3 and 4 were amplified by PCR. 10 μl samples of each primer (stock solution of 10 p.mole/ml) for each exon were mixed and further diluted in distilled water to a concentration of 5 pmole.μl¹ producing a mixed solution of the forward and reverse primers for each exon. The template DNA for the reactions was of two types: control “C” and “RA”. Multiple control and patient samples were available. Control samples were named with a simple number whilst the RA patient DNA samples were given a number with the prefix “A”. So the template samples were 90, 91, 92, 93 and 94 (control) and A11, A12, A13, A14, A16, A17, A18 and A19 (RA). PCR reactions were run in a thermocycler.

The following reaction was set up on ice in a 0.5 ml microfuge tube:

| | | |
|---|---|---|
| DNA | 3 | μl |
| 10x PCR buffer | 5 | μl |
| dNTP mix (200 μM) | 2.5 | μl |
| Primer forward | 2.5 | μl |
| Primer reverse | 2.5 | μl |
| Polymerase | 2 | U |
| Sterile water | to 50 | μl |

The dNTP mix, polymerase and PCR buffer were obtained from Bioline (London, UK). The PCR buffer was NH₄ reaction buffer. DMSO (dimethyl sulphoxide) was also used in some reactions. Magnesium Chloride (50 nM) was added at volumes between 1 and 5 μl. The total volume of the PCR mixture was 50 μl.

Typical PCR programme:

1. 94° C. for 5 minutes. Melting Step.

2. 94° C. for 30 seconds. Melting Step.

3. 53° C.* for 30 seconds. Annealing Step.

4. 72° C. for 1 minute. Elongation Step.

5. Repeat stages 2 to 4 for 30 cycles.

6. 72° C. for 10 minutes. Final Elongation Step.

*Annealing temperature is determined by calculating the melting temperature of the primers and subtracting 5° C. The melting temperature of primers can be calculated using formulae known to the person skilled in the art.

After initial PCR was performed, the lack of immediate success prompted the use to Touch Down PCR. Touch Down PCR uses an atypical protocol where the annealing temperature changes periodically throughout the cycles. It starts at a temperature above the predicted annealing temperature and after a set number of cycles (for example: five) then decreases by 1° C., finally reaching a “Touch Down” annealing temperature in the last set of cycles. The reason for doing this is to examine whether the annealing temperature of the PCR protocol needs to be adjusted. Touch Down PCR has also been shown to reduce spurious priming (Don, R H, Cox, P T, Wainwright, B J, Baker, K and Mattick, J S (1991). “Touchdown” PCR to circumvent spurious priming during gene amplification. Nucleic Acids Research 19: 4008), i.e. non-specific annealing of the primers at incorrect positions, resulting in the production of many different PCR products, thus giving poor (smeared or diffuse) bands on a gel.

### Touch Down PCR Programme:

| | | |
|---|---|---|
| 1 | 94° C. for 5 minutes | |
| 2 | 94° C. for 30 seconds | Repeat steps 2-4 for 5 cycles |
| 3 | 58° C. for 30 seconds | |
| 4 | 72° C. for 1 minute | |
| 5 | 94° C. for 30 seconds | Repeat steps 5-7 for 5 cycles |
| 6 | 56° C. for 30 seconds | |
| 7 | 72° C. for 1 minute | |
| 8 | 94° C. for 30 seconds | Repeat steps 8-10 for 5 cycles |
| 9 | 54° C. for 30 seconds | |
| 10 | 72° C. for 1 minute | |
| 11 | 94° C. for 30 seconds | Repeat steps 11-13 for 5 cycles |
| 12 | 52° C. for 30 seconds | |
| 13 | 72° C. for 1 minute | |
| 14 | 94° C. for 30 seconds | Repeat steps 14-16 for 5 cycles |
| 15 | 50° C. for 30 seconds | |
| 16 | 72° C. for 1 minute | |
| 17 | 94° C. for 30 seconds | Repeat steps 17-19 for 5 cycles |
| 18 | 48° C. for 30 seconds | |
| 19 | 72° C. for 1 minute | |
| 20 | 72° C. for 10 minutes | |

PCR Optimisation. The PCR protocol was optimised to accurately and reliably amplify the Cysteine Dioxygenase exons 2, 3 and 4. Optimisation was carried out using several strategies, most of which involved varying the concentrations of a particular reaction component or experimenting with the presence or absence of an additive. Variables that were controlled included:
Magnesium Chloride concentration.
Template DNA concentration.
Primer concentration.
Primer design.
DMSO addition/omission.
   However, for exon 1 the following PCR programme was used:

| | |
|---|---|
| Stage 1. | 1 cycle of 94° C. for 1 minute |
| Stage 2. | 30 cycles of |
| | 94° C. for 30 seconds |
| | 61° C. for 30 seconds |
| | 72° C. for 1 minute |

Stage 3. 1 cycle of 72° C. for 10 minutes.

and the reaction components were:

| | |
|---|---|
| DNA | 3 μl |
| 10x PCR buffer | 5 μl |
| dNTP mix | 2.5 μl |

Primers for exon 12.5 μl of each of the two primers (each at a concentration of 10 μuM)

| | | |
|---|---|---|
| Polymerase enzyme mix | 2 | U |
| Sterile water | To 50 | μl |

The results of these experiments are assessed by agarose gel electrophoresis and subsequent DNA ‘Plasmid-to-Profile’ sequencing.

PCR amplification of Exon 1. Exon 1 was amplified using PCR primers shown in SEQ ID No. 9 and 10 using standard PCR or touchdown PCR. Exon 1 was also amplified by touchdown PCR using primers SEQ ID No. 9 and 17.

Agarose Gel Electrophoresis. Agarose gels of 1, 2 and 3% were used at different stages of this study. Agarose gels were prepared in TAE buffer. The composition of a 50× stock of TAE buffer is: 242 g Tris Base, 57.1 ml glacial acetic acid, 100 ml 0.5 M EDTA (pH8); made up to 1 litre with distilled water. Agarose gels were prepared according to standard methods (Molecular Cloning: Sambrook, Fritsch and Maniatis, 1989) and included ethidium bromide (Sigma) at standard concentrations.

After completion of the PCR, a 10 μl aliquot of the PCR product was added to 4 μl volume of gel loading dye, in this case Orange G and Glycerol, in a 0.5 ml microfuge tube. These samples were then loaded into a gel. In addition to the PCR products, a mixture of 2 μl loading dye and 5 μl 100 bp DNA ladder (Bioline) was pipetted into the first well of every gel. Gels were run at 125 volts for varying lengths of time (usually at least on hour) for the DNA fragments to migrate sufficiently through the gel for them to be separated by eye when observed under UV light. study The electrophoresis conditions were optimised in order to achieve a suitable resolution and adequate separation of the bands.

PCR Product Purification. Following gel electrophoresis, selected PCR products were purified to isolate the DNA from the other components of the product mixture (denatured Taq DNA polymerase, primers, dNTPs etc.). This was performed using the Qiagen PCR Product Purification Kit (Qiagen, UK). This kit uses a number of buffers (binding buffer, wash buffer and elution buffer) and spin columns to isolate the DNA from the PCR mixture. The kit was used according to manufacturers instructions (provided with kit). This kit elutes the DNA from the PCR product in a 50 μl solution of buffer EB (elution buffer).

Gel Extraction. Another method used to obtain pure DNA samples after PCR was extraction of bands of interest from the agarose gel. Intercalated ethidium bromide allows DNA bands to be identified under UV light. Bands of interest were excised from the gel using a sharp clean scalpel. The excised bands were placed into 1.5 ml microfuge tube and the DNA isolated using the Qiagen Gel Extraction kit (Qiagen, UK). This kit was composed of a number of buffers (similar to that of the PCR purification kit) and spin columns (the same as those from the PCR purification kit). The final purified DNA sample is obtained in a 50 μl volume of elution buffer (buffer EB).

DNA Quantification. DNA sequencing requires that the submitted samples contain a specific amount of DNA (in the present case, between 20 ng and 600 ng of DNA). Therefore the amount of DNA present in the resultant solutions from the purification processes was quantified using UV spectrophotometry at a wavelength of 260 nm. To convert the absorption value into nanograms of DNA per microlitre, the following well-known calculation was performed: UV absorption*50*500=DNA ng μl⁻¹

Preparation of DNA Samples for Sequencing. The correct concentration of DNA for ‘Plasmid to Profile’ sequencing is quoted (by manufacturers) as being between 200 and 600 ng.ml⁻¹. At this stage of the study, part of the experimentation aimed to determine the optimal concentration of DNA in the sequencing solution for optimal results. After experimentation, the samples were diluted to the appropriate concentration of DNA to provide optimal results. Finally, an aliquot (0.64 μl of a 5 p.mole μl⁻¹ solution of primer) of appropriate primer was added to the DNA sample to make the amount of primer in the sequencing sample 3.2p.mole (0.64*5=3.2). This was then submitted to the University of Birmingham Functional Genomics Laboratory for ‘Plasmid to Profile’ sequencing.

Sequence Analysis. Completed sequencing results are obtained from the Functional Genomics Laboratory web site in Chromas™ (Software: Chromas v4.1™ and ChromasPro v1.5™© Telynesium Pty. Ltd. http://www.telynesium.com.au) format (.ab1 files). These can be opened and read using the Chromas software. Within this software it is possible (in conjunction with an internet connection) to perform a BLAST search on the sequence data obtained from the sequencing. The results of this search gives a list of genes and DNA sequences from a number of sources, that show homology with the query sequence (i.e. the data from the plasmid to profile sequencing), arranged in order of homology. These results are referred to as “BLAST hits”. If the sequencing has been fully successful, the top BLAST hit will be “Homo sapiens Cysteine Dioxygenase Exon . . . ”. Sequence alignment diagrams are also included in the BLAST output, showing how close the match is between the query and the reference sequences. This can serve two key uses: firstly, it can show any discrepancies present in the sequencing results (thus can be a measure of sequencing success) and secondly can show any polymorphisms present in the sequenced sample. This is an important aim for this study: to identify any polymorphisms between a control and a disease CDO gene that may be influential in the disease symptoms. If a polymorphism is found that could explain the decreased CDO activity in Rheumatoid Arthritis patients, for example a deleterious polymorphism in an active site domain, this can go some way towards explaining the phenomena observed in vivo i.e. high plasma cysteine levels.

### Results

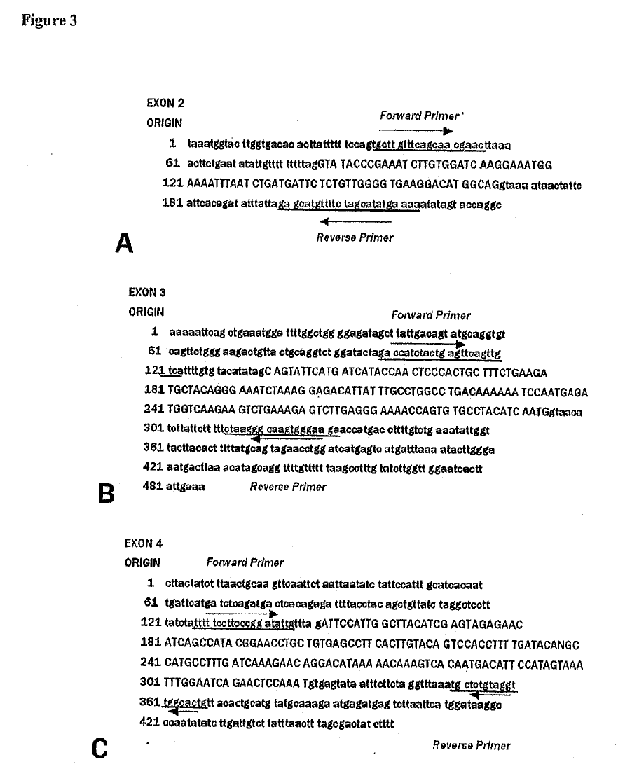

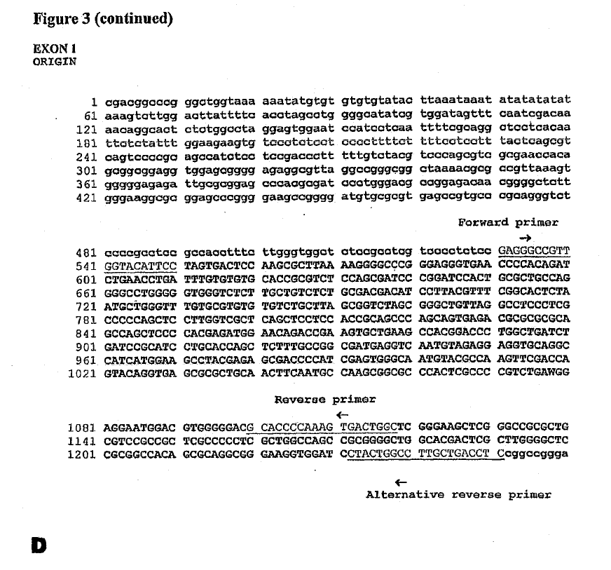

Primer Design. Oligonucleotide primers for the PCR reactions were designed using the primer 3 web tool (Primer 3 internet primer designing tool, http://frodo.wi.mit.edu/cgi-bin/primer3/primer3_www.cgi hosted by Massachusetts Institute of Technology, http://web.mit.edu/index.html). For every set of primers, the intron and exon sequence for that particular section of the gene (e.g. Exon 2, 3 or 4) was uploaded into the Primer 3. Primer 3 then assembled several sets of primers that could be used for each exon. Additional information was also predicted, such as the melting temperatures of the primers, their specificity. The results for the first three sets of primers used and for the exon 1 primers are shown in FIG. 3.

PCR optimisation. PCR is an extremely sensitive technique. It is highly prone to contamination by exogenous DNA and the reactions have many components, the relative proportions of which are very influential on the success of the reaction. These variables were experimented with to determine the optimum concentration. In theory, every component of the reaction can be modified, but for the purposes of this study, only key variables were explored. Below is a list of the constant and variable factors in the PCR reactions.
Constant: Taq DNA polymerase concentration and volume,
   Primer volume,
   Buffer concentration and volume,
   Magnesium Chloride concentration,
   Template DNA volume.
Variable: Primer concentration,
   Primer Sequence,
   Template concentration,
   Magnesium Chloride solution volume,
   DMSO additive present/absent.
      In addition to these parameters, two different types of template DNA were used in this investigation, therefore the PCR conditions for amplification of exons 2, 3 and 4 had to be optimised for both templates. Ideally, the different templates would be successfully amplified under the same conditions, but in this case it was found that the control DNA and rheumatoid arthritis DNA had different requirements for amplification by PCR (see later results).

Agarose Gel Electrophoresis Separation. To assess the success of a PCR reaction, a 10 μl volume of the PCR products was run on an agarose gel. It was found that 2% agarose gels were most appropriate for the PCR products from this study, as they are mainly small molecules (<300 bp) and higher percentage gels give greater separation of the smaller molecules. It was also found that running the gel at a voltage of 120 volts gave the best balance between resolution of the bands and time taken to run the gel; running a gel at this voltage took approximately 75 minutes (data not shown). A gel was considered to have run to completion once the loading dye (Orange G and Glycerol) had traveled approximately three quarters of the length of the gel from the wells.

Initial PCR tests. The initial reactions attempting to amplify the CDO exons performed using typical protocol (see Materials and Methods) and reaction components demonstrated very limited success indicating that amplification would require quite significant optimisation. Examination by electrophoresis showed that no (or very little) DNA was amplified in this reaction, and the DNA ladder provided had to be replaced (the ladder could not be seen on the gel). This lack of DNA on this gel could imply that the control template DNA sample did not contain any DNA. However, this was ruled out as an aliquot of the control template sample was run on a gel to and this showed that it did indeed contain DNA.

In order to explore the possible reason for this initial lack of success, a touch down PCR protocol was devised around the specific primers designed for the three CDO exons. With an average melting temperature of 58° C. (and therefore a PCR annealing temperature of 53° C.), the touch down protocol descended through a series of temperatures surrounding 53° C., touching down at a temperature of 48° C. Below is the protocol.

Touch Down PCR Protocol #01

| | | |
|---|---|---|
| 1 | 94° C. for 5 minutes | |
| 2 | 94° C. for 30 seconds | Repeat steps 2-4 for 5 cycles |
| 3 | 58° C. for 30 seconds | |
| 4 | 72° C. for 1 minute | |
| 5 | 94° C. for 30 seconds | Repeat steps 5-7 for 5 cycles |
| 6 | 56° C. for 30 seconds | |
| 7 | 72° C. for 1 minute | |
| 8 | 94° C. for 30 seconds | Repeat steps 8-10 for 5 cycles |
| 9 | 54° C. for 30 seconds | |
| 10 | 72° C. for 1 minute | |
| 11 | 94° C. for 30 seconds | Repeat steps 11-13 for 10 cycles |
| 12 | 52° C. for 30 seconds | |
| 13 | 72° C. for 1 minute | |
| 14 | 94° C. for 30 seconds | Repeat steps 14-16 for 15 cycles |
| 15 | 50° C. for 30 seconds | |
| 16 | 72° C. for 1 minute | |
| 17 | 94° C. for 30 seconds | Repeat steps 17-19 for 20 cycles |
| 18 | 48° C. for 30 seconds | |
| 19 | 72° C. for 1 minute | |
| 20 | 72° C. for 10 minutes | |

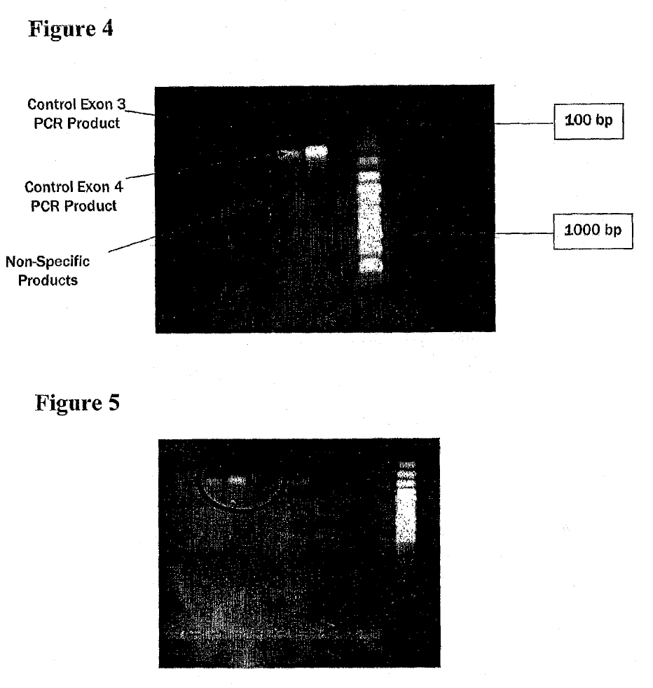

Using this protocol resulted in a partially successful PCR where CDO exons 3 and 4 where amplified, whilst there was no success with exon 2. FIG. 4 shows the gel electrophoresis of the touch down PCR products.

FIG. 4 shows that PCR has been successful for exons 3 and 4, whilst it was unsuccessful for exon 2. On examining the bands, it can be seen that the bands are poorly resolved and demonstrate smearing below the bands themselves. This is caused by the PCR reaction yielding non-specific products, larger than the target product. From this result, it was suggested that the additive dimethylsulphoxide (DMSO) be added to the PCR reactions. FIG. 5 shows the results of the first PCR performed using the DMSO additive; it shows that exons 2, 3, 4 have been amplified. The intensity of the bands indicates the relative concentrations of ethidium bromide and (by implication) the amount of DNA in that area of the gel. The brightest band is that of exon 3, so it can be assumed that PCR has worked best with exon 3 so far.

FIG. 11 shows the PCR products from amplification of exon 1.

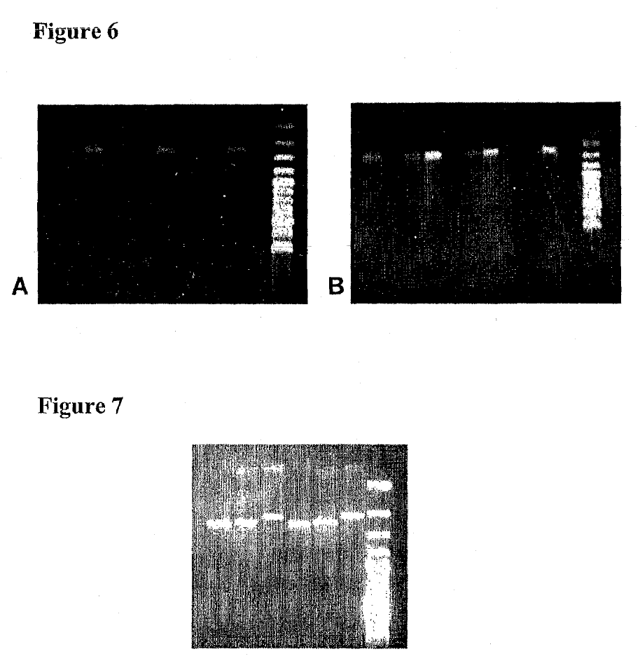

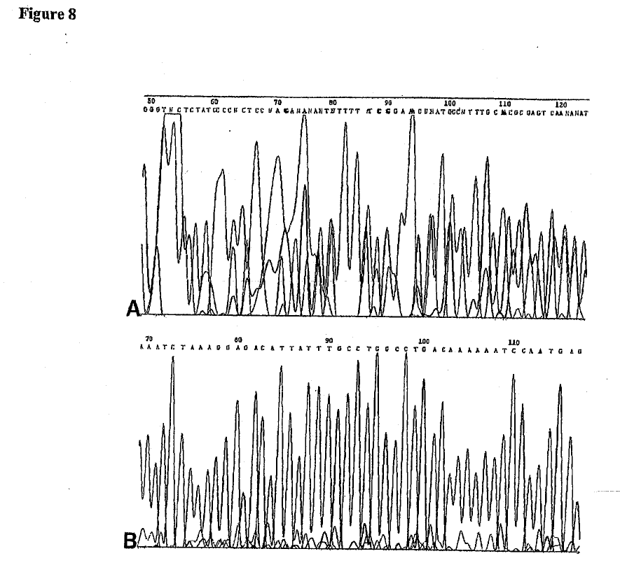

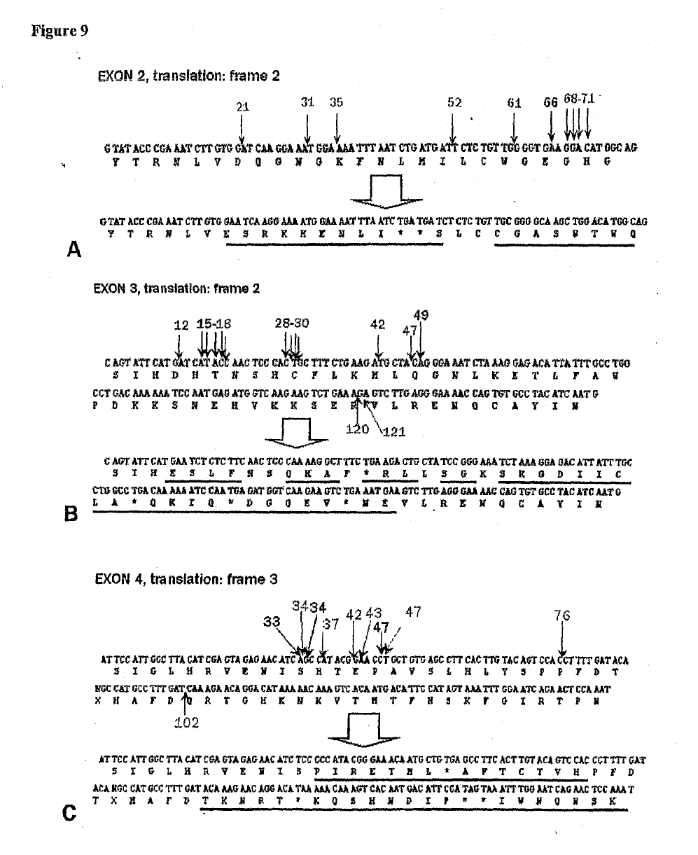

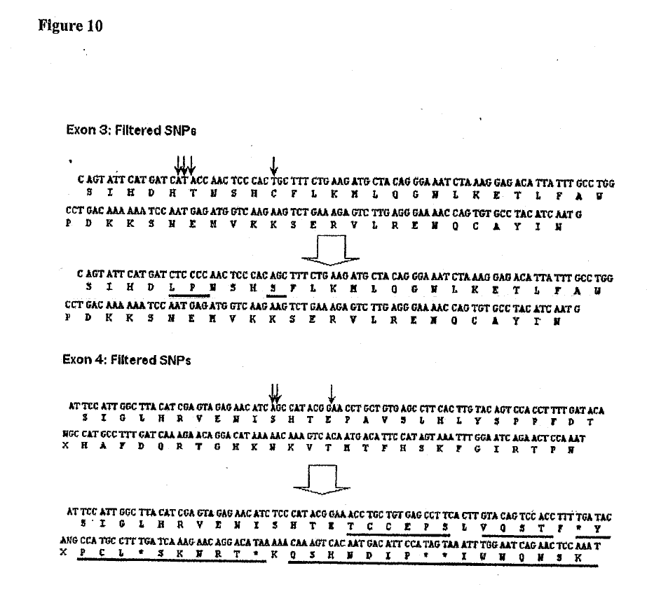

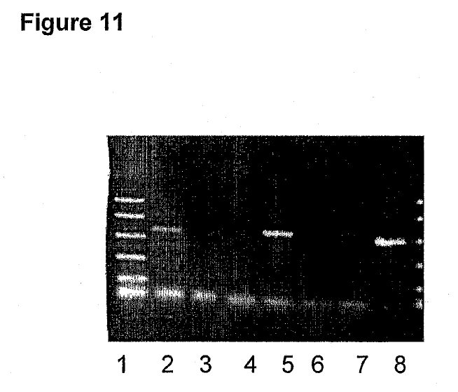

Magnesium Chloride Concentration. Determining the optimum concentration of Magnesium Chloride (MgCl₂) solution in the PCR mixtures was performed by running “Magnesium Gradients”. Here many reactions are set up with differing volumes of MgCl₂ and the results assessed by separating the PCR products on an agarose gel. Typically, the range of MgCl₂ volumes in PCR reactions are between 1-5 μl. FIG. 6 shows the first magnesium gradient on using the control DNA template. From this initial result it can be seen that the amplified DNA bands are marginally brighter (indicating a higher concentration of DNA) in the reactions where the magnesium concentration was 4 μl or 2 μl. However the difference is very slight. There is smearing on most of the bands indicating that the reactions occurred inefficiently, producing an array of varying length products.

Primers. Several pairs of primers had to be designed throughout the course of this study, in order to identify the set that would give the best PCR results. These were all designed using the Primer 3 (Primer 3 internet primer designing tool, http://frodo.wi.mit.edu/cgi-bin/primer3/primer3_www.cgi hosted by Massachusetts Institute of Technology, http://web.mit.edu/index.html) Internet tool. In all five sets of primers were experimented with. Throughout the course of the optimisation experiments, it was possible to optimise the amplification of exons 3 and 4 without having to design new primers, as PCR occurred with sufficient consistency to not require new primers. Other components of the PCR reactions however did need extensive modification.

Exon 2 proved to be the most difficult exon to amplify. Throughout the PCR experiments, exons 3 and 4 showed (to an extent) consistency in their amplification, albeit to varying levels of success. Exon 2 however was often unpredictable in how well the reactions worked, sometimes producing well-resolved tight bands whilst in others producing poorer bands and often no band at all. Two additional sets of primers were designed in an attempt to find consistent PCR success. In designing the primers, it was aimed to try to obtain primers with similar melting temperatures to the primers for exons 3 and 4, so all three exons could be amplified using the same PCR protocol. Exon 1 was amplified using the different PCR protocol as described previously using PCR primers as described in SEQ ID No. 9 and 10.

Template Concentration. Template DNA concentration was examined in exactly the same manner as Magnesium Chloride and primer concentration. A template DNA gradient was run with many reactions using varied concentrations of template DNA. It has already been established that PCR is an extremely sensitive technique (see earlier sections) so theoretically the template concentration should not be a major factor. Template DNA samples were obtained as small aqueous “stock” volumes, from which aliquots were taken and diluted to the desired amount. The stock solutions were also quantified for their DNA content using a spectrophotometer. Dilutions produced from these solutions were ½, 1/10 and 1/20. One experiment was performed to determine the optimum concentration of template DNA in the solution: template DNA was diluted and PCR reactions run using these different dilutions. A previous prediction stated that template concentration should not have a major effect on PCR success, due to the sensitivity of the technique.

TABLE 1
DNA content of the undiluted template DNA samples.
DNA content values are in ng · ml⁻¹.

| Template | UV | |
|---|---|---|
| Sample | Absorption | DNA |
| 90 | 0.057 | 1425 |
| 91 | 0.109 | 2725 |
| 92 | 0.056 | 1400 |
| 93 | 0.045 | 1125 |
| 94 | 0.082 | 2050 |
| A11 | 0.026 | 650 |
| A12 | 0.03 | 750 |
| A13 | 0.035 | 875 |
| A14 | 0.02 | 500 |
| A16 | 0.021 | 525 |
| A17 | 0.023 | 575 |
| A18 | 0.028 | 700 |
| A19 | 0.021 | 525 |

Touchdown PCR. As mentioned earlier, Touchdown PCR was employed early in the study to achieve some early success with the amplification experiments (see FIG. 7). Because of this early success, Touchdown PCR was investigated again as a possible means of amplifying exons 2, 3 and 4 on one PCR protocol. Using the optimised PCR components (see table 1) two Touchdown PCR protocols were devised: one starting at 55° C., dropping 1° C. every set of cycles; and the other starting at 54° C. dropping 2° C. every cycle. These reactions produced mixed results, one set of reactions worked very well, efficiently amplifying all three exons, whilst others failed to work at all. It was considered best to concentrate on optimising different reactions for every exon rather trying to tailor one protocol for all three exons. One successful Touchdown PCR result is shown in FIG. 7. T his was an extremely promising technique.

Summary of Optimised Polymerase Chain Reaction Conditions. From all the experiments performed in this study, an optimised scheme for amplification of Homo sapiens Cysteine Dioxygenase Exons 2, 3 and 4 has been devised. These details are given to in Table 2. To verify these findings, PCR reactions were set up using the optimised conditions for amplification of all exons from a number of control and RA patient DNA samples. In a majority of cases the PCR was successful. The products from these reactions were then purified and submitted for sequencing.

TABLE 2
Summary Table of the Optimised conditions for amplification of Homo
sapiens CDO exons 2, 3 and 4.

| | | | | | Primers | |
|---|---|---|---|---|---|---|
| | | | | | Average | Successful |
| Tem- | | Prim- | | | Melting | Annealing |
| plate | Exon | ers | [MgCl2] | DMSO | Temperature | Temperature |
| Control | 2 | Set 3 | 4 | X | 55.35 | 50 |
| | 3 | Set 1 | 2 | ✓ | 57.65 | 53 |
| | 4 | Set 1 | 2 | X | 57.00 | 51.6 |
| RA | 2 | Set 3 | 4 | X | 55.35 | 50 |
| | 3 | Set 1 | 3 | ✓ | 57.65 | 53 |
| | 4 | Set 1 | 3 | X | 57.00 | 51.6 |

Exon 1 was amplified using the exon-1 specific primers and the different reaction mixture and parameters as described previously.

PCR Product Purification. PCR products were isolated and purified using the Qiagen PCR purification kit as described previously. 3.2 p.mole of the purified product were submitted for Plasmid to Profile sequencing. After initial experiments with this technique it became apparent that the purification process may have isolated all the DNA from the PCR reaction, but the DNA collected was too heterogeneous to give accurate sequencing results. All sequencing results obtained by submitting DNA purified in this manner were too poor to give accurate sequencing results. A poor resolution of the sequence resulted in no significant matches being found through BLAST searches.

These findings prompted a more discreet system to be employed for the isolation and purification of the correct DNA oligonucleotides amplified by PCR. Gel Extraction from agarose gel slab was selected as a replacement for PCR product purification. This yielded dramatically improved results from the sequencing.

Gel Extraction. Gel Extraction yielded good purity of DNA (as shown by gel electrophoresis of the extracted and purified DNA) and improved sequencing results. One minor drawback is that because of the reduced DNA concentration, bands from a gel extraction sample were often faint and difficult to observe under UV (data not included due to poor images). Most importantly the sequencing results obtained from gel extracted DNA were of far higher quality than those obtained through PCR product purification. Examples of sequences obtained from these two methods are shown in FIG. 8.

Sequencing and Polymorphism Analysis. Initial sequencing attempts met with very limited success for a number of reasons, mainly contamination and impurity of template DNA (amplified from patient sample) and primers. The principal technique for purification of the PCR products was with the Qiagen PCR purification kit using spin columns. The sequencing results using this technique were of poor quality with little usable sequence. Employing the gel extraction method yielded far better results, and after a little further optimisation of the PCR conditions, gave good sequencing results of suitable quality to allow a sequence comparison. The sequence data was compared with the reference sequence to identify any Single Nucleotide Polymorphisms (SNPs), single base pair differences in the sequences, from which a record of possible SNPs was constructed. There are a significant number of polymorphisms between the control and RA sequences. To represent the data in more visual form, the polymorphisms for Exons 2, 3 and 4 have been highlighted on the sequence in FIG. 9.

TABLE 3A

| | | Fre- | | | | |
|---|---|---|---|---|---|---|
| Exon | SNP | quency | C | RA | Type | Comment |
| Exon 2 | +18 N-T | 2 | | ✓ | Substitution | Potential False |
| | | | | | | Positive |
| Exon 2 | +19 N-G | 2 | | ✓ | Substitution | Potential False |
| | | | | | | Positive |
| Exon 2 | +20 N-G | 1 | | ✓ | Substitution | Potential False |
| | | | | | | Positive |
| Exon 2 | +21 A-NBP | 1 | | ✓ | Addition | Potential SNP |
| Exon 2 | +31 A-NBP | 1 | ✓ | | Addition | Potential SNP |
| Exon 2 | +35 T-NBP | 1 | ✓ | | Addition | Potential SNP |
| Exon 2 | +40 N-T | 1 | | ✓ | Substitution | Potential False |
| | | | | | | Positive |
| Exon 2 | +52 C-NBP | 1 | ✓ | | Addition | Potential SNP |
| Exon 2 | +61 C-NBP | 1 | ✓ | | Addition | Potential SNP |
| Exon 2 | +66 C-G | 1 | ✓ | | Substitution | Potential SNP |
| Exon 2 | +68 G-T | 1 | ✓ | | Substitution | Potential SNP |
| Exon 2 | +69 C-NBP | 1 | ✓ | | Addition | Potential SNP |
| Exon 2 | +70 T-NBP | 1 | ✓ | | Addition | Potential SNP |
| Exon 2 | +71 G-NBP | 1 | ✓ | | Addition | Potential SNP |
| Exon 2 | +77 N-NBP | 1 | ✓ | | Addition | Potential False |
| | | | | | | Positive |
| Exon 2 | +84 T-NBP | 1 | ✓ | | Addition | Not in exon |

TABLE 3B

| | | Fre- | | | | |
|---|---|---|---|---|---|---|
| Exon | SNP | quency | C | RA | Type | Comment |
| Exon 3 | +12 A-NBP | 1 | ✓ | | Addition | Potential SNP |
| Exon 3 | +15 N-A | 1 | | ✓ | Substitution | Potential False |
| | | | | | | Positive |
| Exon 3 | +15 T-A | 2 | ✓ | ✓ | Substitution | Potential SNP |
| Exon 3 | +16 C-T | 2 | | ✓ | Substitution | Potential SNP |
| Exon 3 | +16 N-T | 1 | | ✓ | Substitution | Potential False |
| | | | | | | Positive |
| Exon 3 | +17 N-A | 3 | ✓ | ✓ | Substitution | Potential False |
| | | | | | | Positive |
| Exon 3 | +17 T-A | 1 | | ✓ | Substitution | Potential SNP |
| Exon 3 | +17 C-A | 3 | ✓ | ✓ | Substitution | Potential SNP |
| Exon 3 | +18 T-C | 1 | | ✓ | Substitution | Potential SNP |
| Exon 3 | +18 T-NBP | 1 | ✓ | | Addition | Potential SNP |
| Exon 3 | +19 N-C | 1 | ✓ | | Substitution | Potential False |
| | | | | | | Positive |
| Exon 3 | +28 A-C | 1 | | ✓ | Substitution | Potential SNP |
| Exon 3 | +29 A-NBP | 1 | | ✓ | Addition | Potential SNP |
| Exon 3 | +29 A-T | 2 | | ✓ | Substitution | Potential SNP |
| Exon 3 | +30 G-NBP | 2 | ✓ | | Addition | Potential SNP |
| Exon 3 | +42 C-NBP | 1 | ✓ | | Addition | Potential SNP |
| Exon 3 | +46 N-A | 1 | | ✓ | Substitution | Potential False |
| | | | | | | Positive |
| Exon 3 | +47 T-NBP | 1 | | ✓ | Addition | Potential SNP |
| Exon 3 | +49 C-A | 1 | | ✓ | Substitution | Potential SNP |
| Exon 3 | +52 N-G | 1 | | ✓ | Substitution | Potential False |
| | | | | | | Positive |
| Exon 3 | +52 N-NBP | 3 | ✓ | ✓ | Addition | Potential False |
| | | | | | | Positive |
| Exon 3 | +53 N-NBP | 1 | | ✓ | Addition | Potential False |
| | | | | | | Positive |
| Exon 3 | +92 N-A | 1 | ✓ | | Substitution | Potential False |
| | | | | | | Positive |
| Exon 3 | +113 N-T | 1 | | ✓ | Substitution | Potential False |
| | | | | | | Positive |
| Exon 3 | +120 T-NBP | 1 | | ✓ | Addition | Potential SNP |
| Exon 3 | +121 A-NBP | 1 | ✓ | | Addition | Potential SNP |

TABLE 3C

| | | Fre- | | | | |
|---|---|---|---|---|---|---|
| Exon | SNP | quency | C | RA | Type | Comment |
| Exon 4 | +33 C-A | 1 | | ✓ | Substitution | Potential SNP |
| Exon 4 | +33 T-A | 4 | | ✓ | Substitution | Potential SNP |
| Exon 4 | +33 N-A | 1 | ✓ | ✓ | Substitution | Potential False |
| | | | | | | Positive |
| Exon 4 | +34 C-G | 5 | | ✓ | Substitution | Potential SNP |
| Exon 4 | +34 T-G | 1 | ✓ | ✓ | Substitution | Potential SNP |
| Exon 4 | +34 N-G | 2 | ✓ | ✓ | Substitution | Potential False |
| | | | | | | Positive |
| Exon 4 | +34 C-NBP | 1 | | ✓ | Addition | Potential SNP |
| Exon 4 | +35 N-G | 1 | | ✓ | Substitution | Potential False |
| | | | | | | Positive |
| Exon 4 | +37 C-NBP | 1 | | ✓ | Addition | Potential SNP |
| Exon 4 | +42 G-NBP | 1 | | ✓ | Addition | Potential SNP |
| Exon 4 | +43 N-NBP | 2 | ✓ | | Addition | Potential False |
| | | | | | | Positive |
| Exon 4 | +43 G-NBP | 1 | ✓ | | Addition | Potential SNP |
| Exon 4 | +43 A-NBP | 4 | | ✓ | Addition | Potential SNP |
| Exon 4 | +44 N-NBP | 1 | | ✓ | Addition | Potential False |
| | | | | | | Positive |
| Exon 4 | +46 A-C | 2 | ✓ | | Substitution | Potential SNP |
| Exon 4 | +47 A-C | 1 | | ✓ | Substitution | Potential SNP |
| Exon 4 | +47 N-C | 2 | ✓ | | Substitution | Potential False |
| | | | | | | Positive |
| Exon 4 | +47 A-NBP | 1 | | ✓ | Addition | Potential SNP |
| Exon 4 | +48 N-T | 1 | ✓ | | Substitution | Potential False |
| | | | | | | Positive |
| Exon 4 | +76 C-NBP | 1 | | ✓ | Addition | Potential SNP |
| Exon 4 | +102 A-NBP | 1 | ✓ | | Addition | Potential SNP |

Tables 3 A, B and C. Summary Tables of SNPs found in each of exons 2, 3 and 4.

Likewise, for exon 1 the inventors identified 55 mutations. Through further analysis they identified three of these as SNPs.

It should be noted that for the substitutions shown in Tables 3A, B and C the terminology is the reverse of that previously used. For example, in Table 3B, the substitution at position 15 of Exon 3 is described as +15 T-A. Using the standard terminology (including the terminology used previously in this application) this would read 15A-T. Tables 3A, B and C are internally consistent and consistent with each other. For clarity, throughout the main body of this application the standard terminology has been used, i.e. the preferred polymorphisms are described using standard terminology.

The results show that there is a considerable distribution of SNPs throughout the exons. Exon 2 shows a wide spread distribution of SNP loci, whilst in exon 4 they are confined to far less sites. However, the data collected from the sequencing must be filtered, as it is likely that a proportion of these potential SNPs are merely incidences of the sequencing equipment being unable to determine the nucleotide at a particular position. In addition to the potential false positives, other potential SNPs identified could be in misleading positions due to the polymorphisms upstream of them in the DNA sequence. In an attempt to filter the results for higher quality data, all potential SNPs containing an “N” have been designated “Potential False Positives” and the remaining polymorphisms will be assumed legitimate results. These “Potential SNPs” (see table 3) have been mapped onto the recorded DNA sequence of the three exons and translated to investigate the consequences for the protein derived from these polymorphic exons. FIG. 9 shows that the potential SNPs would cause dramatic and (almost certainly) deleterious changes to the amino acid sequence. In most cases almost half the amino acid sequence is different to the original. In vivo, an individual with all these polymorphisms in their CDO exons would almost certainly possess inefficient or retarded CDO enzyme.

It should be noted that the term ‘Potential False Positive’ does not mean that the inventors have disregarded these results as being irrelevant for RA detection. Instead it is meant to emphasise that those results designated ‘Potential SNP’ are more likely to indicate the presence of RA.

Filtering Results. In each of the samples sequenced, none contained all the SNPs identified here. The frequency at which particular SNPs arise is significant, as this can indicate highly polymorphic loci within the CDO exons. The frequencies of the SNPs are given in table 4. To give a more relevant representation of the in vivo situation, only the most frequently occurring SNPs (i.e. more than 1 occurrence in this study) alone have been examined and represented in FIG. 10.

TABLE 4
Reduced table of Potential RA SNPs occurring more than
once in data set.

| | | | | Frequency |
|---|---|---|---|---|
| Exon | SNP | Position | Frequency in controls | in RA patients |
| 1 | A (ins) | 673 | 0/30 | 10/22 |
| 1 | G (ins) | 697 | 2/30 | 9/22 |
| 1 | C-A | 1,103 | /4/30 | 9/22 |

| | | Posi- | Fre- | | | | |
|---|---|---|---|---|---|---|---|
| Exon | SNP | tion | quency | C | RA | Type | Comment |
| 3 | A-T | 15 | 2 | + | + | Substitution | Potential SNP |
| 3 | T-C | 16 | 2 | − | + | Substitution | Potential SNP |
| 3 | A-C | 17 | 3 | + | + | Substitution | Potential SNP |
| 3 | T-A | 29 | 2 | − | + | Substitution | Potential SNP |
| 4 | A-T | 33 | 4 | − | + | Substitution | Potential SNP |
| 4 | G-C | 34 | 5 | − | + | Substitution | Potential SNP |
| 4 | A (ins) | 43 | 4 | − | + | Addition | Potential SNP |

### Discussion

The inventors have been able to successfully optimise a PCR protocol for the amplification and sequencing of Homo sapiens Cysteine Dioxygenase Exons 2, 3 and 4. This was achieved through a series of PCR experiments that were able to show which conditions allowed the various exons to be amplified and sequenced in a relatively short period of time. The inventors have also generated a PCR protocol suitable for the amplification and sequencing of Homo Sapiens Cysteine Dioxygenase Exon 1. Various polymorphisms were identified in exons 1, 3 and 4.

The Effect of Compounds on CDO Activity. The inventors realise that CDO may be a key enzyme involved in rheumatoid arthritis. The inventors had already identified that TNF-α and TGF-β affect CDO activity (Wilkinson L. J. and Waring R. H. 2002 Supra).

Analysis of the effects of IFN-γ and TNF-α were assayed using the methods described in Wilkinson L. J. and Waring R. H. (Toxicol in vitro (2002), pages 481-483).

The effect of Interferon-γ on CDO expression is shown in Table 5:

| IFN-gamma | CDO expression (% of | TNF-alpha | CDO expression (% of |
|---|---|---|---|
| (ng/ml) | 0 ng/ml expression) | (ng/ml) | 0 ng/ml expression) |
| 0 | 100.0 | 0.0 | 100.0 |
| 1.0 | 110.3 ± 17.9 | 0.01 | 88.5 ± 16.7 |
| 2.5 | 148.8 ± 8.8 | 0.05 | 70.1 ± 7.9 |
| 5.0 | 132.5 ± 23.6 | 0.10 | 51.8 ± 12.2 |
| 7.5 | 157.1 ± 6.9 | 0.25 | 42.0 ± 18.3 |
| 10.0 | 161.6 ± 7.8 | 0.50 | 35.6 ± 12.0 |

This shows that Interferon-γ may be used to increase CDO expression, and therefore increase the amount of sulphate available, thus reducing rheumatoid arthritis symptoms. Furthermore, it may be used with compounds known to increase the production of sulphate, for example, cysteine, methionine, D-penicillamine, N-acetyl cysteine, γ-glutamylcysteine, interleukin-4 and interleukin-10, S-carboxymethyl cysteine, S-methyl cysteine to further improve the treatment of rheumatoid arthritis.

1. A method for diagnosing a cysteine dioxygenase (CDO)-mediated condition, which method comprises:
(i) obtaining a sample from an individual;
(ii) detecting the presence or absence of a variant CDO, or a nucleic acid encoding a CDO variant and/or a variant of a CDO regulatory region; a
(iii) determining the status of the individual by reference to polymorphism in the CDO gene and/or its regulatory region.

2. A method for diagnosing one or more polymorphisms in the CDO gene, or its regulatory sequence in a human comprising determining the sequence of a nucleic acid molecule encoding at least a portion of the CDO gene and/or a CDO regulatory region and determining the status of the human by reference to polymorphism in the CDO gene and/or its regulatory region.

3. A method according to claim 1, wherein the polymorphism is:

| Exon | Position | SNP | Type |
|---|---|---|---|
| Exon 1: | +673 | A | insertion |
| Exon 1: | +697 | G | insertion |
| Exon 1: | +1103 | C-A | substitution |
| Exon 3: | +15 | A-T | substitution |
| Exon 3: | +16 | T-C | substitution |
| Exon 3: | +17 | A-C | substitution |
| Exon 3: | +29 | T-A | substitution |
| Exon 3: | +30 | G | addition |
| Exon 4: | +33 | A-T | substitution |
| Exon 4: | +34 | G-C | substitution |
| Exon 4: | +43 | A | addition, or |
| Exon 4: | +46 | C-A | substitution |

4. The method of claim 1, wherein the status provides an indication that the human has decreased CDO concentrations and/or CDO having decreased enzymatic activity.

5. The method of claim 1, for identification of the propensity of the individual or human to develop rheumatoid arthritis and/or to develop side-effects with one or more drugs selected from D-penicillamine and gold thiomalate.

6. The method of claim 1, wherein the region of nucleic acid containing a polymorphism is amplified by polymerase chain reaction (PCR) prior to identifying the polymorphism.

7. The method of claim 6, wherein the PCR is real time PCR.

8. The method of claim 1, wherein a nucleic acid sequence is determined by a method selected from ARMS-allele specific amplification, allele specific hybridisation, oligonucleotide ligation assay and restriction fragment length polymorphism.

9. An allele specific probe or primer capable of detecting a CDO gene or CDO regulatory sequence polymorphism.

10. An allele specific probe or primer according to claim 9 capable of detecting a polymorphism at one or more of:

| Exon | Position | SNP | Type |
|---|---|---|---|
| Exon 1: | +673 | A | insertion |
| Exon 1: | +697 | G | insertion |
| Exon 1: | +1103 | C-A | substitution |
| Exon 3: | +15 | A-T | substitution |
| Exon 3: | +16 | T-C | substitution |
| Exon 3: | +17 | A-C | substitution |
| Exon 3: | +29 | T-A | substitution |
| Exon 3: | +30 | G | addition |
| Exon 4: | +33 | A-T | substitution |
| Exon 4: | +34 | G-C | substitution |
| Exon 4: | +43 | A | addition, or |
| Exon 4: | +46 | C-A | substitution |

11. A diagnostic kit comprising one or more probes or primers according to claim 9.

12. An isolated nucleic acid of at least 5 bases long encoding a CDO or CDO-regulatory polymorphism selected from:
(i)

| Exon | Position | SNP | Type |
|---|---|---|---|
| Exon 1: | +673 | A | insertion |
| Exon 1: | +697 | G | insertion |
| Exon 1: | +1103 | C-A | substitution |
| Exon 3: | +15 | A-T | substitution |
| Exon 3: | +16 | T-C | substitution |
| Exon 3: | +17 | A-C | substitution |
| Exon 3: | +29 | T-A | substitution |
| Exon 3: | +30 | G | addition |
| Exon 4: | +33 | A-T | substitution |
| Exon 4: | +34 | G-C | substitution |
| Exon 4: | +43 | A | addition |
| Exon 4: | +46 | C-A | substitution |

and
(ii) sequences complementary to such sequences.

13. An isolated nucleic acid molecule according to claim 12 which encodes CDO or a fragment thereof.

14. An isolated CDO protein obtainable from a nucleic acid molecule according to claim 13.

15. An antibody capable of specifically binding a protein according to claim 14, but not to a CDO protein that does not contain the CDO polymorphism.

16. A method of determining the propensity of an individual to a CDO-mediated condition comprising:
(i) Obtaining a sample from the individual; and
(ii) Detecting the presence of a CDO containing the polymorphism using an antibody according to claim 15.

17. A method of determining the effect of a compound on CDO activity comprising:
(i) Providing a sample of white blood cells;
(ii) Contacting the white blood cells with a CDO substrate and the compound; and
(iii) Determining the effect of the compound on the conversion of the substrate to a detectable product by CDO in the white blood cells.

18. A method of identifying a drug candidate for the treatment of rheumatoid arthritis, comprising the use of a method according to claim 17.

19. A method of treating rheumatoid arthritis comprising administering a pharmaceutically effective amount of interferon-γ (IFN-γ).

20. A method according to claim 19, wherein the rheumatoid arthritis is non-HTLV-1 associated rheumatoid arthritis.

21. The method of claim 19, wherein the IFN-γ is administered in combination with at least one of: cysteine, methionine, D-penicillamine, N-acetyl cysteine, γ-glutamylcysteine, interleukin-4 or interleukin-10.

22-23. (canceled)

24. A pharmaceutically acceptable composition comprising IFN-γ and at least one of cysteine, methionine, D-penicillamine, N-acetyl cysteine, γ-glutamylcysteine, S-carboxy methyl cysteine, S-methyl cysteine, interleukin-4 or interleukin-10.

25. The method of claim 2, wherein the polymorphism is:

| Exon | Position | SNP | Type |
|---|---|---|---|
| Exon 1: | +673 | A | insertion |
| Exon 1: | +697 | G | insertion |
| Exon 1: | +1103 | C-A | substitution |
| Exon 3: | +15 | A-T | substitution |
| Exon 3: | +16 | T-C | substitution |
| Exon 3: | +17 | A-C | substitution |
| Exon 3: | +29 | T-A | substitution |
| Exon 3: | +30 | G | addition |
| Exon 4: | +33 | A-T | substitution |
| Exon 4: | +34 | G-C | substitution |
| Exon 4: | +43 | A | addition, or |
| Exon 4: | +46 | C-A | substitution |

26. The method of claim 2, wherein the status provides an indication that the human has decreased CDO concentrations and/or CDO having decreased enzymatic activity.

27. The method of claim 2, for identification of the propensity of the individual or human to develop rheumatoid arthritis and/or to develop side-effects with one or more drugs selected from D-penicillamine and gold thiomalate.

28. The method of claim 2, wherein the region of nucleic acid containing a polymorphism is amplified by PCR prior to identifying the polymorphism.

29. The method of claim 28, wherein the PCR is real time PCR.

30. The method of claim 2 wherein a nucleic acid sequence is determined by a method selected from ARMS-allele specific amplification, allele specific hybridization, oligonucleotide ligation assay and restriction fragment length polymorphism.
